(19) United States
(12) Patent Application Publication
(10) Pub. No.: US 2025/0255874 A1
(43) Pub. Date: Aug. 14, 2025

(54) Methods for Treating Immune Thrombocytopenia By Administering (R)-2-[3-[4-Amino-3-(2-Fluoro-4-Phenoxy-Phenyl)Pyrazolo[3,4-D]Pyrimidin-1-YL]Piperidine-1-Carbonyl]-4-Methyl-4-[4-(Oxetan-3-YL)Piperazin-1-YL]Pent-2-Enentrile

(71) Applicant: Principia Biopharma Inc., Bridgewater, NJ (US)

(72) Inventors: Ahmed Daak, Concord, MA (US); Umer Khan, Orlando, FL (US)

(21) Appl. No.: 19/194,148

(22) Filed: Apr. 30, 2025

Related U.S. Application Data
(63) Continuation of application No. PCT/US2023/078211, filed on Oct. 30, 2023.
(60) Provisional application No. 63/386,094, filed on Dec. 5, 2022.
(60) Provisional application No. 63/420,864, filed on Oct. 31, 2022.

(51) Int. Cl. A61K 31/519; A61K 9/28; A61P 7/00

(52) U.S. Cl. CPC A61K 31/519; A61K 9/28; A61P 7/00

(57) ABSTRACT

Methods for treating immune thrombocytopenia comprising administering at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof are disclosed.

Disclosed herein are methods for treating immune thrombocytopenia. BTK inhibitors and pharmaceutical compositions comprising the same are also disclosed.

Immune thrombocytopenia, commonly referred to as ITP, is a rare autoimmune disease with a heterogeneous pathophysiology that causes high risk for bleeding, excessive bruising, and fatigue, as well as the potential for life threatening intracranial bleeding due to destruction of platelets. ITP is characterized by immune-mediated (e.g., autoantibody-mediated) platelet destruction and impaired platelet production, resulting in thrombocytopenia, a predisposition to bleeding associated with morbidity and mortality, and an adverse impact on patient quality of life (QOL).

Current therapies for adults with ITP include initial treatment with intravenous immunoglobulin (IVIG) and corticosteroids, and subsequent treatment with splenectomy, thrombopoietin receptor agonists (TPO-RAs), rituximab, fostamatinib, and immunosuppressive therapies (such as, e.g., mycophenolate mofetil (MMF) and cyclosporine). In general, pharmacotherapy (e.g., corticosteroids, IVIG, or anti-D immunoglobulin therapy) is used for symptomatic patients with low platelet counts for reducing platelet destruction. While most patients respond initially to corticosteroids, the rate of continued remission is low. Second line therapies for ITP include rituximab and splenectomy, which are associated with risk of sepsis and immune suppression. Additionally, thrombopoietin (TPO) mimetics (Bussel 2007) are approved for the treatment of patients with chronic ITP who have not had sufficient responses to corticosteroids, IVIG, or splenectomy. Novel, safe, and effective oral treatments to maintain platelet counts in ITP patients would represent a significant therapeutic advantage over current standard of care. By way of non-limiting example, unmet needs in relapsed and refractory ITP include: improving remission rates and durability; avoiding rapid increase of platelet counts/thrombosis risk; steroid-free regimens; and a tolerable and safe therapy that ensures good patient QOL. Thus, there is a need for novel oral therapies for treating ITP, including relapsed and refractory ITP, that address some or all of these limitations of existing therapeutic modalities.

Bruton's agammaglobulinemia tyrosine kinase (BTK) is an essential signaling element downstream of the B-cell receptor (BCR), Fc-gamma receptor (FcγR), and Fc-epsilon receptor (FcεR). BTK is a non-receptor tyrosine kinase and a member of the TEC family of kinases. BTK is essential to B cell lineage maturation, and inhibition of BTK activity in cells produces phenotypic changes consistent with blockade of the BCR. Illustratively, BTK inhibition results in the down-regulation of various B-cell activities, including cell proliferation, differentiation, maturation, and survival, and the up-regulation of apoptosis.

Rather than acting in an “on/off switch” manner, BTK may be best viewed as an immune function “modulator” (Crofford U et al., 2016; Pal Singh S et al., 2018). Important insights into BTK function come from loss of function analyses in humans and mice. Individuals with loss of function mutations in the BTK gene develop X-linked agammaglobulinemia (XLA), characterized by a complete absence of circulating B cells and plasma cells, and very low levels of immunoglobulins of all classes (Tsukada 1993, Vetrie 1993). This indicates the potential for BTK inhibition to suppress production of autoantibodies thought to be important in the development of autoimmune diseases, such as ITP.

While BTK is not expressed in T cells, natural killer cells, or plasma cells and has no traceable direct functions in T cells or plasma cells (Sideras and Smith 1995; Mohamed et al., 2009), the enzyme regulates the activation of other hematopoietic cells, such as B cells, monocytes, basophils, mast cells, macrophages, neutrophils, and platelets. For example, BTK plays a role in the activation of neutrophils, which are key players in the inflammatory response that contributes to wound healing but may also cause tissue damage (Volmering S et al., 2016).

Accordingly, a selective BTK inhibitor has the potential to target multiple pathways involved in inflammation and autoimmunity, including, but not limited to: blocking BCR signaling; inhibiting plasma cell differentiation and antibody production; blocking IgG-mediated FcγR activation, phagocytosis, and inflammatory mediators in monocytes or macrophages; blocking IgE-mediated FcεR activation and degranulation in mast cells or basophils; and inhibiting activation, adhesion, recruitment, and oxidative burst in neutrophils. Based on these effects, a selective BTK inhibitor may block the initiation and progression of various inflammatory diseases and mitigate tissue damage resulting from these diseases. Although individuals with loss of function mutations in the BTK gene have decreased humoral immunity and are susceptible to pyogenic bacterial and enterovirus infections, requiring treatment with intravenous immunoglobulin, inhibition of BTK in individuals with an intact immune system is not predicted to produce similar susceptibility to infection.

Several orally administered BTK inhibitors (BTKi), including ibrutinib (PCI-32765) and spebrutinib (CC-292), are currently marketed or in clinical development for a range of indications (Lee A et al., 2017). For example, ibrutinib has provided further clinical validation of the BTK target and was recently approved for human use in mantle cell lymphoma, Waldenstrom's macroglobulinemia, and chronic lymphocytic leukemia by the U.S. Food and Drug Administration (FDA). Ibrutinib has also demonstrated activity in other hematological malignancies (Wang 2013; Byrd 2013, Imbruvica Package Insert, 2015). In addition, CC-292 has been reported to be well tolerated in a healthy volunteer population at doses which provide 100% occupancy of the BTK enzyme (Evans 2013). Furthermore, evobrutinib recently demonstrated efficacy for multiple sclerosis in a Phase 2 trial (Montalban X et al., 2019). Other BTKi compounds are in clinical development for various immune-mediated disorders, such as pemphigus (NCT02704429), rheumatoid arthritis (NCT03823378, NCT03682705, NCT03233230), and asthma (NCT03944707) (Montalban X et al., 2019; Norman P 2016; Tam C S et al., 2018; Crawford J J et al., 2018; Min T K et al., 2019; Gillooly K M 2017; Nadeem A et al., 2019).

While covalent BTKi, such as ibrutinib and acalabrutinib, improved on the selectivity issues that plagued many first-generation kinase inhibitors, these inhibitors are typically irreversible, causing permanent modification of both on- and off-target kinases and side effects such as thrombocytopenia, anemia, platelet aggregation, and hepatotoxicity (RITUXAN Prescribing Information, 2018; Drug Record Kinase Inhibitors, 2019; Khan Y et al., 2019; Paydas S, 2019; IMBRUVICA, 2013; Rigg R A et al., 2016; Tang C P S et al., 2018). Thus, there is a need for treatment modalities for immune-mediated diseases such as ITP, based on BTKi with reduced side effects.

Compound (I) is a BTK inhibitor of the following structure:

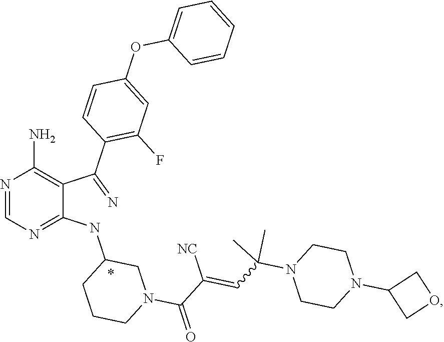

wherein *C is a stereochemical center. See PCT Publication No. WO 2014/039899, which is incorporated herein by reference, e.g., Example 31.
(R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, having the following structure:

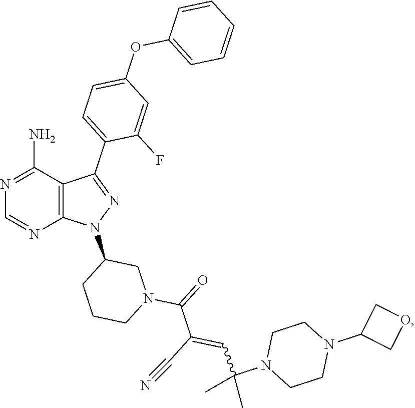 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

is also known as PRN1008 and rilzabrutinib. This compound has been disclosed in several patent publications, such as, e.g., PCT Publication Nos. WO 2014/039899, WO 2015/127310, WO 2016/100914, WO 2016/105531, and WO 2018/005849, the contents of each of which are incorporated by reference herein.

Rilzabrutinib is a novel, highly selective, small molecule inhibitor of non-T cell white blood cell signaling via B-cell receptor, FcγR, and/or FcεR signaling of the BTK pathway. Rilzabrutinib functions as a reversible covalent BTK inhibitor and forms both a non-covalent and a covalent bond with its target, allowing for enhanced selectivity and extended inhibition with low systemic exposure. In comparison to first and second generation BTKi, rilzabrutinib has shown minimal cross-reactivity with other molecules and is low risk for off-target effects (Smith P F et al., 2017). Importantly, rilzabrutinib's reversible binding minimizes the likelihood of permanently modified peptides (Serafimova IM 2012). In addition, rilzabrutinib shows improved kinase selectivity relative to the covalent BTK inhibitor ibrutinib. Preclinical studies in a broad kinase enzyme inhibition panel showed that 1 μM rilzabrutinib achieved >90% inhibition of just 6 of 251 kinases sharing a common cysteine in their active site. By contrast, 1 μM ibrutinib inhibited 21 kinases. Rilzabrutinib's IC50 values were 1.3 nM for BTK, 0.8 nM for tyrosine protein kinase TEC, 1.0 nM for bone marrow tyrosine kinase on chromosome X (BMX), 1.2 nM for receptor-like kinase (RLK), 6.3 nM for B cell lymphocyte kinase (BLK), and 11 nM for ERBB4. Further preclinical assays with rilzabrutinib showed that binding to BTK persisted while that for other TEC family members decayed rapidly over time.

Rilzabrutinib has shown encouraging results for the treatment of immune-mediated diseases. Rilzabrutinib is the most advanced BTKi in development for an autoimmune disease (Phase 3, NCT03762265). In humans, rilzabrutinib is rapidly absorbed following oral administration, with a fast half-life (3-4 h) and variable pharmacokinetics (PK) (Smith P F et al., 2017).

In Phase 1 studies of rilzabrutinib with 114 healthy volunteers, target BTK occupancy levels were safely and consistently exceeded, suggesting rilzabrutinib may be highly effective in treating autoimmune diseases. Moreover, preclinical and clinical PK/PD data showed that treatment effects endured even after the compound was cleared from circulation, consistent with an extended target residence time (Hill R et al., 2015) and high occupancy rate (>90% within four hours) (Smith P F et al., 2015).

Rilzabrutinib has also demonstrated a favorable safety profile. Based on preclinical reproductive toxicity studies, rilzabrutinib is not expected to harm fetal development or male fertility. In a Phase 1 study in healthy volunteers, the most commonly reported adverse events were gastrointestinal adverse events, including nausea/vomiting and diarrhea. No serious adverse events or deaths were reported, and no participants discontinued treatment due to an adverse event (Smith P F 2017).

There is preliminary evidence to support the role of BTK inhibition in patients with autoimmune cytopenias (Rogers 2016, Montillo 2017), where sequential episodes of severe autoimmune hemolytic anemia and ITP ceased after initiation of treatment with ibrutinib, a BTK/EGFR/ITK inhibitor, in patients with chronic lymphatic leukemia (CLL). Additionally, and pertinent to the treatment of ITP, rilzabrutinib treatment in vitro profoundly inhibits human B cell activation and blocks antibody (IgG, IgE) mediated activation of immune cells via Fc receptor signaling. In nonclinical studies, rilzabrutinib demonstrates a significant dose dependent reduction of platelet-loss (consumption) in a mouse model of ITP. Rilzabrutinib also shows rapid and significant anti-inflammatory effects in a rat collagen-induced arthritis model, a rat antibody-mediated Arthus model, spontaneous canine pemphigus foliaceus, and human pemphigus vulgaris (PV).

Although there is promising preliminary evidence to support the role of rilzabrutinib in patients with ITP, a patient's response to ITP therapy generally remains challenging to predict, and neither a durable response nor long-term remission is guaranteed. In particular, there are few predictive markers for the efficacy of currently available ITP therapies, making it difficult to identify patients who will successfully respond to new therapies. Consequently, predicting the response to current therapies is crucial because it helps optimize the use of current and future therapies.

A recent study of an ongoing phase 1/2 trial (NCT03395210) on the use of rilzabrutinib in patients with ITP identified several possible predictors of a successful response to rilzabrutinib. The study, which examined a total of 11 baseline clinical variables in 45 patients who initiated 400 mg of rilzabrutinib twice daily, revealed that patients with a shorter duration of ITP and/or no prior use of thrombopoietin receptor agonists or rituximab were more likely to respond to treatment with rilzabrutinib. Moreover, patients with no prior use of thrombopoietin receptor agonists were more likely to be early responders to rilzabrutinib treatment.

Disclosed herein are methods for treating immune thrombocytopenia (ITP) in a human patient in need thereof who has not received prior treatment with a thrombopoietin receptor agonist (TPO-RA) or rituximab comprising administering to the human patient who has not received prior treatment with a TPO-RA or rituximab a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day for a treatment period, wherein the human patient in need thereof has at least one characteristic chosen from: a platelet count of count <33,000/μL on two occasions no less than 7 days apart in the 15 days prior to beginning study treatment; and a history of response (two or more platelet counts ≥50,000/μL with an increase of ≥20,000/μL) to at least one prior line of therapy. In some embodiments, the human patient in need thereof has a platelet count of <33,000/μL on two occasions no less than 7 days apart in the 15 days prior to beginning study treatment. In some embodiments, the human patient in need thereof has a history of response (two or more platelet counts ≥50,000/μL with an increase of ≥20,000/μL) to at least one prior line of therapy. In some embodiments, the human patient in need thereof has a platelet count of <33,000/μL on two occasions no less than 7 days apart in the 15 days prior to beginning study treatment and has a history of response (two or more platelet counts ≥50,000/μL with an increase of ≥20,000/μL) to at least one prior line of therapy.

Also disclosed herein are methods for treating immune thrombocytopenia (ITP) in a human patient in need thereof who has not received prior treatment with a thrombopoietin receptor agonist (TPO-RA) comprising administering to the human patient who has not received prior treatment with a TPO-RA a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day for a treatment period, wherein the human patient in need thereof has at least one characteristic chosen from: a platelet count of count <33,000/μL on two occasions no less than 7 days apart in the 15 days prior to beginning study treatment; and a history of response (two or more platelet counts ≥50,000/μL with an increase of ≥20,000/μL) to at least one prior line of therapy. In some embodiments, the human patient in need thereof has a platelet count of <33,000/μL on two occasions no less than 7 days apart in the 15 days prior to beginning study treatment. In some embodiments, the human patient in need thereof has a history of response (two or more platelet counts ≥50,000/μL with an increase of ≥20,000/μL) to at least one prior line of therapy. In some embodiments, the human patient in need thereof has a platelet count of <33,000/μL on two occasions no less than 7 days apart in the 15 days prior to beginning study treatment and has a history of response (two or more platelet counts ≥50,000/μL with an increase of ≥20,000/μL) to at least one prior line of therapy.

Also disclosed herein are methods for treating immune thrombocytopenia (ITP) in a human patient in need thereof who has not received prior treatment with rituximab comprising administering to the human patient who has not received prior treatment with rituximab a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day for a treatment period, wherein the human patient in need thereof has at least one characteristic chosen from: a platelet count of count <33,000/μL on two occasions no less than 7 days apart in the 15 days prior to beginning study treatment; and a history of response (two or more platelet counts ≥50,000/μL with an increase of ≥20,000/μL) to at least one prior line of therapy. In some embodiments, the human patient in need thereof has a platelet count of <33,000/μL on two occasions no less than 7 days apart in the 15 days prior to beginning study treatment. In some embodiments, the human patient in need thereof has a history of response (two or more platelet counts ≥50,000/μL with an increase of ≥20,000/μL) to at least one prior line of therapy. In some embodiments, the human patient in need thereof has a platelet count of <33,000/μL on two occasions no less than 7 days apart in the 15 days prior to beginning study treatment and has a history of response (two or more platelet counts ≥50,000/μL with an increase of ≥20,000/μL) to at least one prior line of therapy.

### BRIEF DESCRIPTION OF DRAWINGS

FIG. 1 shows the number of patients initiating rilzabritinib at each dose, the number of patients who completed the main study, and the number of patients who entered the long-term extension.

FIG. 2 shows a summary of intrapatient dose-escalation levels with rilzabrutinib.

FIG. 3 shows IBLS Bleeding Scale scores at baseline (Cycle 1, Day 1) and week 24/end of study for patients who completed 24 weeks of rilzabrutinib (n=34). Bleeding symptoms were grouped by site of bleeding and scored based on grade from lowest (0) to highest (3-4; grade 5 for fatal bleeding), as defined and standardized by the ITP International Working Group. There were no grade 3, 4, or 5 bleeding events at baseline or week 24/EOS; therefore, bars are not shown for these grades.

FIG. 4 shows the median platelet count over time for patients initiating rilzabrutinib at all doses, including 400 mg bid (N=60), separated by responders and nonresponders.

FIG. 5 shows the median platelet count over time for patients initiating rilzabrutinib at 400 mg bid (n=45), separated by responders and nonresponders.

FIG. 6 shows the median platelet count over time for patients initiating rilzabrutinib at all doses, including 400 mg bid (N=60).

FIG. 7 shows the median platelet count over time for patients initiating rilzabrutinib at 400 mg bid (n=45).

FIG. 8 shows rilzabrutinib dose and platelet response over time.

FIG. 9 shows the percentage of patients in each subgroup who achieved ≥2 consecutive platelet counts, separated by at least 5 days, of ≥50,000/μL, and increased ≥20,000/μL from baseline, without requiring rescue medication in the 4 weeks prior to the latest elevated platelet count.

FIG. 10 shows a summary of continuous variables by outcome for nonresponders vs. responders (top) and nonearly responders vs. early responders (bottom).

FIG. 11 shows the number of patients initiating rilzabritinib at each dose, the number of patients who achieved the primary platelet response, and the number of patients who were early responders to rilzabrutinib.

### DEFINITIONS

Unless otherwise stated, the following terms used in the specification and claims are defined for the purposes of this Application and have the following meanings. All undefined technical and scientific terms used in this Application have the meaning as commonly understood by one of ordinary skill in the art to which this disclosure belongs.

As used herein, “a” or “an” entity refers to one or more of that entity; for example, a compound refers to one or more compounds or at least one compound unless stated otherwise. As such, the terms “a” (or “an”), “one or more”, and “at least one” can be used interchangeably herein.

As used herein, the term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%. With regard to specific values, it should be understood that specific values described herein for subject populations (e.g., the subject of the described clinical trial) represent median, mean, or statistical numbers, unless otherwise provided. Accordingly, aspects of the present disclosure requiring a particular value in a subject are supported herein by population data in which the relevant value is assessed to be a meaningful delimitation on the subject population.

As used herein, the term “active pharmaceutical ingredient” or “therapeutic agent” (“API”) refers to a biologically active compound.

As used herein, the term “approved treatment” refers to a medication that has received regulatory authorization, in any country, for its intended use.

As used herein, the terms “administer,” “administering,” or “administration” herein refer to providing, giving, dosing, and/or prescribing by either a health practitioner or an authorized agent and/or putting into, taking, or consuming by the patient or person himself or herself. For example, “administration” of an API to a patient refers to any route (e.g., oral delivery) of introducing or delivering the API to the patient. Administration includes self-administration and administration by another.

As used herein, the terms “baseline platelet count” or “baseline” refer to an average platelet count obtained by determining the mean of two platelet counts taken on two occasions no less than 7 days apart in the 15 days prior to beginning treatment and a third count taken on the first day of the study. If any of these three counts is missing, the “baseline platelet count” or “baseline” is the average of the other counts. As used herein, “BID” and “bid” are used interchangeably to refer to twice a day.

As used herein, “immune thrombocytopenia” (ITP) encompasses or at least also refers to other terms commonly used such as idiopathic thrombocytopenia and idiopathic thrombocytopenic purpura. There are three main types of ITP: Acute (short term), persistent, and chronic (long term). Acute ITP lasts less than three months, persistent ITP lasts 3-12 months, and chronic ITP lasts for at least one year.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other during a treatment period. Unless specified otherwise, the two or more compounds, agents, or active pharmaceutical ingredients may be administered on different schedules during the treatment period, such as, e.g., with one or more compounds, agents, or active pharmaceutical ingredients being administered once a day and one or more other compounds, agents, or active pharmaceutical ingredients being administered twice a day.

As used herein, an amount expressed in terms of “mg of [X]” refers to the total amount in milligrams of [X], i.e., the free base. In some embodiments, rilzabrutinib may be administered as a pharmaceutically acceptable salt of rilzabrutinib, in which case an amount expressed in terms of “mg of rilzabrutinib” refers to the total amount in milligrams of rilzabrutinib, i.e., the free base, plus the equivalent amount of one or more pharmaceutically acceptable salts of rilzabrutinib based on the weight of free base therein. For example, “400 mg of at least one compound chosen from rilzabrutinib and pharmaceutically acceptable salts thereof” includes 400 mg of rilzabrutinib and a concentration of one or more pharmaceutically acceptable salts of rilzabrutinib equivalent to 400 mg of rilzabrutinib.

As used herein, a “pharmaceutically acceptable carrier or excipient” means a carrier or an excipient that is useful in preparing a pharmaceutical composition that is generally safe, and neither biologically nor otherwise undesirable, such as, e.g., a carrier or an excipient that is acceptable for mammalian pharmaceutical use.

As used herein, the term “pharmaceutically acceptable salt” refers to a salt form, e.g., an acid addition salt, of an active pharmaceutical agent that is pharmaceutically acceptable and that possesses the desired pharmacological activity of the API of which the salt is made. Pharmaceutically acceptable salts are well known in the art and include those derived from suitable inorganic and organic acids. Such salts include, but are not limited to, salts formed with inorganic acids such as hydrochloric acid, hydrobromic acid, sulfuric acid, phosphoric acid, and the like; or formed with organic acids such as formic acid, acetic acid, propionic acid, hexanoic acid, lactic acid, malonic acid, succinic acid, malic acid, maleic acid, fumaric acid, tartaric acid, citric acid, benzoic acid, mandelic acid, methanesulfonic acid, ethanesulfonic acid, 1,2-ethanedisulfonic acid, benzenesulfonic acid, 4-toluenesulfonic acid, and the like. S. M. Berge et al. describes pharmaceutically acceptable salts in detail in J. Pharmaceutical Sciences, 1977, 66, 1-19.

As used herein, the terms “PRN1008,” “rilzabrutinib,” “(R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile” and “2-[(3R)-3-[4-amino-3-(2-fluoro-4-phenoxy-phenyl)pyrazolo[3,4-d]-pyrimidin-1-yl]piperidine-1-carbonyl]-4-methyl-4-[4-(oxetan-3-yl)piperazin-1-yl]pent-2-enenitrile” are used interchangeably to refer to a compound having the structure:

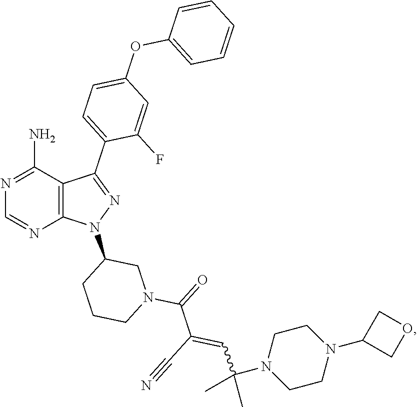 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

which is also referred to as 2-[(3R)-2-[4-amino-3-(2-fluoro-4-phenoxy-phenyl)pyrazolo[3,4-d]pyrimidin-1-yl]piperdine-1-carbonyl]-4-methyl-4[4-(oxetan-3-yl)piperazin-1-yl]-(E and Z)-pent-2-enenitrile; (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile; 1-piperidinepropanenitrile, 3-[4-amino-3-(2-fluoro-4-phenoxyphenyl)-1H-pyrazolo[3,4-d]pyrimidin-1-yl]-a-[2-methyl-2-[4-(3-oxetanyl)-1-piperazinyl]propylidene]-p-oxo-, (3R)-; (EZ)-2-[(3R)-3-[4-amino-3-(2-fluoro-4-phenoxyphenyl)pyrazolo[3,4-d]pyrimidin-1-yl]piperidine-1-carbonyl]-4-methyl-4[4-(oxetan-3-yl)piperazin-1-yl]pent-2-enenitrile; and also by the International Nonproprietary Names for Pharmaceutical Substances (INN) as published by the World Health Organization (https://cdn.who.int/media/docs/default-source/international-nonpropietary-names-(inn)/1121.pdf!sfcrns=69617906_15&download=true) having the following structure:

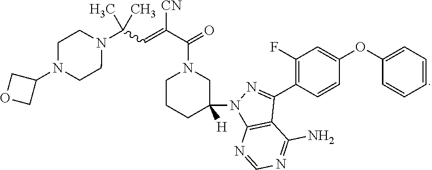 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1 ; "."]

The compound of Formula (I) includes E and Z isomers, as indicated by the wavy bond in the structure shown above. The compound of Formula (I) may be present as a salt form.

As isomer of rilzabrutinib may contain the corresponding (Z) isomer as an impurity in less than about 1% by weight; a dose of the (Z) isomer of rilzabrutinib may contain the corresponding (E) isomer as an impurity in less than about 1% by weight. When rilzabrutinib is denoted as a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, it means that the amount of (E) or (Z) isomer in the mixture is greater than about 10% by weight. In some embodiments, the molar ratio of (E) to (Z) isomer is 9:1 rilzabrutinib or a pharmaceutically acceptable salt thereof may also be referred to herein as a “drug,” “active agent,” “a therapeutically active agent,” or “API.”

As used herein, “QD” and “qd” are used interchangeably to refer to once a day.

As used herein, the term “therapeutically effective amount” refers to that an of a compound that produces the desired effect for which it is administered (e.g., improvement in ITP or a symptom of ITP, or lessening the severity of ITP or a symptom of ITP). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the term “treat,” “treating,” or “treatment,” when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art.

Some embodiments of the present disclosure relate to methods for treating immune thrombocytopenia (ITP) in a human patient in need thereof who has not received prior treatment with a thrombopoietin receptor agonist (TPO-RA) or rituximab comprising administering to the human patient who has not received prior treatment with a TPO-RA or rituximab a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day for a treatment period, wherein the human patient in need thereof has at least one characteristic chosen from: a platelet count of count <33,000/μL on two occasions no less than 7 days apart in the 15 days prior to beginning study treatment; and a history of response (two or more platelet counts ≥50,000/μL with an increase of ≥20,000/μL) to at least one prior line of therapy. In some embodiments, the human patient in need thereof has a platelet count of <33,000/μL on two occasions no less than 7 days apart in the 15 days prior to beginning study treatment. In some embodiments, the human patient in need thereof has a history of response (two or more platelet counts ≥50,000/μL with an increase of ≥20,000/μL) to at least one prior line of therapy. In some embodiments, the human patient in need thereof has a platelet count of <33,000/μL on two occasions no less than 7 days apart in the 15 days prior to beginning study treatment and has a history of response (two or more platelet counts ≥50,000/μL with an increase of ≥20,000/μL) to at least one prior line of therapy.

In some embodiments, the TPO-RA is chosen from recombinant thrombopoietin (rTPO), romiplostim, eltrombopag, and avatrombopag. In some embodiments, the TPO-RA is rTPO. In some embodiments, the TPO-RA is romiplostim. In some embodiments, the TPO-RA is avatrombopag. In some embodiments, the TPO-RA is avatrombopag.

In some embodiments, the human patient has not received prior treatment with rituximab.

In some embodiments, the human patient has a history of response (two or more platelet counts ≥50,000/μL with an increase of ≥20,000/μL) to at least one prior line of therapy. In some embodiments, at least one prior therapy is chosen from a splenectomy, intravenous immunoglobin (IVIG), corticosteroids, anti-D immunoglobulin therapy, and immunosuppressive drugs. In some embodiments, the at least one prior therapy is a splenectomy. In some embodiments, the at least one prior therapy is IVIG. In some embodiments, the at least one prior therapy is corticosteroids. In some embodiments, the at least one prior therapy is anti-D immunoglobulin therapy. In some embodiments, the at least one prior therapy is an immunosuppressive drug.

In some embodiments, the human patient achieves at least one platelet count of at least 20,000/μL above a baseline platelet count, wherein the baseline platelet count is the mean of two platelet counts taken on two occasions no less than 7 days apart in the 15 days prior to beginning treatment and a third count taken on the first day of the study. In some embodiments, the human patient achieves at least two platelet counts of at least 20,000/μL above the baseline platelet count. In some embodiments, the human patient achieves at least two consecutive platelet counts of at least 20,000/μL above the baseline platelet count, for example at least 30,000/μL above the baseline platelet count.

In some embodiments, the human patient achieves at least two consecutive platelet counts of at least 20,000/μL above the baseline platelet count that are separated by at least five days, for example at least seven days. In some embodiments, the human patient achieves at least two consecutive platelet counts of at least 20,000/μL above the baseline platelet count that are separated by at least five days. In some embodiments, the human patient achieves at least two consecutive platelet counts of at least 20,000/μL above the baseline platelet count that are separated by at least seven days.

In some embodiments, the human patient achieves a platelet count of at least 50,000/μL. In some embodiments, the human patient achieves at least two platelet counts of at least 50,000/μL. In some embodiments, the human patient achieves at least two consecutive platelet counts of at least 50,000/μL.

In some embodiments, the human patient achieves at least two consecutive platelet counts of at least 50,000/μL that are separated by at least five days, for example at least seven days. In some embodiments, the human patient achieves at least two consecutive platelet counts of at least 50,000/μL that are separated by at least five days. In some embodiments, the human patient achieves at least two consecutive platelet counts of at least 50,000/μL that are separated by at least seven days.

In some embodiments, the human patient achieves at least one platelet count of at least 50,000/μL within eight days of initiating treatment.

In some embodiments, the human patient does not receive rescue medication during the four weeks prior to the most recent platelet count of at least 50,000/μL.

In some embodiments, the human patient has had ITP for six or less years. In some embodiments, the human patient has had ITP for five or less years. In some embodiments, the human patient has had ITP for four or less years. In some embodiments, the human patient has had ITP for three or less years. In some embodiments, the human patient has had ITP for two or less years. In some embodiments, the human patient has had ITP for less than a year.

In some embodiments, the human patient had ITP for at least 1 year prior to the start of the treatment period. In some embodiments, the human patient had ITP for at least 2 years prior to the start of the treatment period. In some embodiments, the human patient had ITP for at least 3 years prior to the start of the treatment period. In some embodiments, the human patient had ITP for at least 4 years prior to the start of the treatment period. In some embodiments, the human patient had ITP for at least 5 years prior to the start of the treatment period. In some embodiments, the human patient had ITP for at least 6 years prior to the start of the treatment period. In some embodiments, the human patient had ITP for at least 7 years prior to the start of the treatment period. In some embodiments, the human patient had ITP for at least 8 years prior to the start of the treatment period. In some embodiments, the human patient had ITP for at least 9 years prior to the start of the treatment period.

In some embodiments, the human patient had ITP for at least 10 years prior to the start of the treatment period. In some embodiments, the human patient had ITP for at least 20 years prior to the start of the treatment period. In some embodiments, the human patient had ITP for at least 30 years prior to the start of the treatment period. In some embodiments, the human patient had ITP for at least 40 years prior to the start of the treatment period. In some embodiments, the human patient had ITP for at least 50 years prior to the start of the treatment period.

In some embodiments, the human patient has a history of taking at least one prior ITP therapy prior to the start of the treatment period. In some embodiments, the human patient has a history of taking at least two prior ITP therapies prior to the start of the treatment period. In some embodiments, the human patient has a history of taking at least three prior ITP therapies prior to the start of the treatment period. In some embodiments, the human patient has a history of taking at least four prior ITP therapies prior to the start of the treatment period. In some embodiments, the human patient has a history of taking at least five prior ITP therapies prior to the start of the treatment period.

In some embodiments, the human patient has platelet counts between 2,000/μL and 33,000/μL on two occasions no less than 7 days apart and within 15 days prior to the start of the treatment period.

In some embodiments, the human patient had a splenectomy prior to the start of the treatment period.

In some embodiments, the human patient has a history of response to at least one prior line of therapy, wherein at least one prior therapy is chosen from a splenectomy, intravenous immunoglobin (IVIG), corticosteroids, anti-D immunoglobulin therapy, and immunosuppressive drugs. In some embodiments, the human patient has a history of response to a splenectomy. In some embodiments, the human patient has a history of response to IVIG. In some embodiments, the human patient has a history of response to corticosteroids. In some embodiments, the human patient has a history of response to anti-D immunoglobulin therapy. In some embodiments, the human patient has a history of response to immunosuppressive drugs.

In some embodiments, the human patient has a history of taking IVIG. In some embodiments, the human patient has a history of taking corticosteroids. In some embodiments, the human patient has a history of taking anti-D immunoglobulin therapy. In some embodiments, the human patient has a history of taking at least one immunosuppressive drug. In some embodiments, the human patient has a history of taking at least one immunosuppressive drug selected from fostamatinib, mycophenolate mofetil (MMF), and cyclosporine. In some embodiments, the human patient has a history of taking fostamatinib. In some embodiments, the human patient has a history of taking mycophenolate mofetil (MMF). In some embodiments, the human patient has a history of taking cyclosporine.

In some embodiments, the response to the prior ITP therapy comprised a platelet count of ≥50,000/μL.

In some embodiments, the human patient has primary ITP. In some embodiments, the human patient has secondary ITP. In some embodiments, the human patient does not have chronic ITP. In some embodiments, the human patient has persistent ITP. In some embodiments, the human patient has chronic ITP. In some embodiments, the human patient has relapsing ITP. In some embodiments, the human patient has refractory ITP.

In some embodiments, the treatment period is at least 8 days. In some embodiments, the treatment period is at least 28 days. In some embodiments, the treatment period is at least 84 days. In some embodiments, the treatment period is at least 169 days.

In some embodiments, the method comprises administering to the human patient 400 mg of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day. In some embodiments, the at least one compound consists of at least one compound chosen from the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof. In some embodiments, the at least one compound consists of at least one compound chosen from the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof. In some embodiments, the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt of the foregoing.

In other embodiments, the method comprises administering to the human patient 400 mg of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile twice a day. In some embodiments, the at least one compound is the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile. In some embodiments, the at least one compound is the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile. In some embodiments, the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.

In some embodiments, the at least one compound is orally administered to the human patient. In some embodiments, the at least one compound is administered to the human patient in the form of at least one tablet. In some embodiments, the at least one compound is administered with water. In some embodiments, the at least one compound is administered with food. In some embodiments, the at least one compound is administered without food.

Some embodiments of the present disclosure relate to methods for treating immune thrombocytopenia (ITP) in a human patient in need thereof who has not received prior treatment with a thrombopoietin receptor agonist (TPO-RA) comprising administering to the human patient who has not received prior treatment with a TPO-RA a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day for a treatment period, wherein the human patient in need thereof has at least one characteristic chosen from: a platelet count of count <33,000/μL on two occasions no less than 7 days apart in the 15 days prior to beginning study treatment; and a history of response (two or more platelet counts ≥50,000/μL with an increase of ≥20,000/μL) to at least one prior line of therapy. In some embodiments, the human patient in need thereof has a platelet count of <33,000/μL on two occasions no less than 7 days apart in the 15 days prior to beginning study treatment. In some embodiments, the human patient in need thereof has a history of response (two or more platelet counts ≥50,000/μL with an increase of ≥20,000/μL) to at least one prior line of therapy. In some embodiments, the human patient in need thereof has a platelet count of <33,000/μL on two occasions no less than 7 days apart in the 15 days prior to beginning study treatment and has a history of response (two or more platelet counts ≥50,000/μL with an increase of ≥20,000/μL) to at least one prior line of therapy.

In some embodiments, the TPO-RA is chosen from recombinant thrombopoietin (rTPO), romiplostim, eltrombopag, and avatrombopag. In some embodiments, the TPO-RA is rTPO. In some embodiments, the TPO-RA is romiplostim. In some embodiments, the TPO-RA is avatrombopag. In some embodiments, the TPO-RA is avatrombopag.

In some embodiments, the human patient has not received prior treatment with rituximab.

In some embodiments, the human patient has a history of response (two or more platelet counts ≥50,000/μL with an increase of ≥20,000/μL) to at least one prior line of therapy. In some embodiments, at least one prior therapy is chosen from a splenectomy, intravenous immunoglobin (IVIG), corticosteroids, anti-D immunoglobulin therapy, and immunosuppressive drugs. In some embodiments, the at least one prior therapy is a splenectomy. In some embodiments, the at least one prior therapy is IVIG. In some embodiments, the at least one prior therapy is corticosteroids. In some embodiments, the at least one prior therapy is anti-D immunoglobulin therapy. In some embodiments, the at least one prior therapy is an immunosuppressive drug.

In some embodiments, the human patient achieves at least one platelet count of at least 20,000/μL above a baseline platelet count, wherein the baseline platelet count is the mean of two platelet counts taken on two occasions no less than 7 days apart in the 15 days prior to beginning treatment and a third count taken on the first day of the study. In some embodiments, the human patient achieves at least two platelet counts of at least 20,000/μL above the baseline platelet count. In some embodiments, the human patient achieves at least two consecutive platelet counts of at least 20,000/μL above the baseline platelet count, for example at least 30,000/μL above the baseline platelet count.

In some embodiments, the human patient achieves at least two consecutive platelet counts of at least 20,000/μL above the baseline platelet count that are separated by at least five days, for example at least seven days. In some embodiments, the human patient achieves at least two consecutive platelet counts of at least 20,000/μL above the baseline platelet count that are separated by at least five days. In some embodiments, the human patient achieves at least two consecutive platelet counts of at least 20,000/μL above the baseline platelet count that are separated by at least seven days.

In some embodiments, the human patient achieves a platelet count of at least 50,000/μL. In some embodiments, the human patient achieves at least two platelet counts of at least 50,000/μL. In some embodiments, the human patient achieves at least two consecutive platelet counts of at least 50,000/μL.

In some embodiments, the human patient achieves at least two consecutive platelet counts of at least 50,000/μL that are separated by at least five days, for example at least seven days. In some embodiments, the human patient achieves at least two consecutive platelet counts of at least 50,000/μL that are separated by at least five days. In some embodiments, the human patient achieves at least two consecutive platelet counts of at least 50,000/μL that are separated by at least seven days.

In some embodiments, the human patient achieves at least one platelet count of at least 50,000/μL within eight days of initiating treatment.

In some embodiments, the human patient does not receive rescue medication during the four weeks prior to the most recent platelet count of at least 50,000/μL.

In some embodiments, the human patient has had ITP for six or less years. In some embodiments, the human patient has had ITP for five or less years. In some embodiments, the human patient has had ITP for four or less years. In some embodiments, the human patient has had ITP for three or less years. In some embodiments, the human patient has had ITP for two or less years. In some embodiments, the human patient has had ITP for less than a year.

In some embodiments, the human patient has a history of taking at least one prior ITP therapy prior to the start of the treatment period. In some embodiments, the human patient has a history of taking at least two prior ITP therapies prior to the start of the treatment period. In some embodiments, the human patient has a history of taking at least three prior ITP therapies prior to the start of the treatment period. In some embodiments, the human patient has a history of taking at least four prior ITP therapies prior to the start of the treatment period. In some embodiments, the human patient has a history of taking at least five prior ITP therapies prior to the start of the treatment period.

In some embodiments, the human patient has platelet counts between 2,000/μL and 33,000/μL on two occasions no less than 7 days apart and within 15 days prior to the start of the treatment period.

In some embodiments, the human patient had a splenectomy prior to the start of the treatment period.

In some embodiments, the human patient has a history of taking rituximab prior to the start of the treatment period.

In some embodiments, the human patient has a history of response to at least one prior line of therapy, wherein at least one prior therapy is chosen from a splenectomy, intravenous immunoglobin (IVIG), corticosteroids, anti-D immunoglobulin therapy, and immunosuppressive drugs. In some embodiments, the human patient has a history of response to a splenectomy. In some embodiments, the human patient has a history of response to IVIG. In some embodiments, the human patient has a history of response to corticosteroids. In some embodiments, the human patient has a history of response to anti-D immunoglobulin therapy. In some embodiments, the human patient has a history of response to immunosuppressive drugs.

In some embodiments, the human patient has a history of taking IVIG. In some embodiments, the human patient has a history of taking corticosteroids. In some embodiments, the human patient has a history of taking anti-D immunoglobulin therapy. In some embodiments, the human patient has a history of taking at least one immunosuppressive drug. In some embodiments, the human patient has a history of taking at least one immunosuppressive drug selected from fostamatinib, mycophenolate mofetil (MMF), and cyclosporine. In some embodiments, the human patient has a history of taking fostamatinib. In some embodiments, the human patient has a history of taking mycophenolate mofetil (MMF). In some embodiments, the human patient has a history of taking cyclosporine.

In some embodiments, the response to the prior ITP therapy comprised a platelet count of ≥50,000/μL.

In some embodiments, the human patient has primary ITP. In some embodiments, the human patient has secondary ITP. In some embodiments, the human patient does not have chronic ITP. In some embodiments, the human patient has persistent ITP. In some embodiments, the human patient has chronic ITP. In some embodiments, the human patient has relapsing ITP. In some embodiments, the human patient has refractory ITP.

In some embodiments, the treatment period is at least 8 days. In some embodiments, the treatment period is at least 28 days. In some embodiments, the treatment period is at least 84 days. In some embodiments, the treatment period is at least 169 days.

In some embodiments, the method comprises administering to the human patient 400 mg of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day. In some embodiments, the at least one compound consists of at least one compound chosen from the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof. In some embodiments, the at least one compound consists of at least one compound chosen from the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof. In some embodiments, the at least one compound consists of a mixture of (R) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt of the foregoing.

In other embodiments, the method comprises administering to the human patient 400 mg of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile twice a day. In some embodiments, the at least one compound is the (R) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile. In some embodiments, the at least one compound is the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile. In some embodiments, the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.

In some embodiments, the at least one compound is orally administered to the human patient. In some embodiments, the at least one compound is administered to the human patient in the form of at least one tablet. In some embodiments, the at least one compound is administered with water. In some embodiments, the at least one compound is administered with food. In some embodiments, the at least one compound is administered without food.

Some embodiments of the present disclosure relate to methods for treating immune thrombocytopenia (ITP) in a human patient in need thereof who has not received prior treatment with rituximab comprising administering to the human patient who has not received prior treatment with rituximab a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day for a treatment period, wherein the human patient in need thereof has at least one characteristic chosen from: a platelet count of count <33,000/μL on two occasions no less than 7 days apart in the 15 days prior to beginning study treatment; and a history of response (two or more platelet counts ≥50,000/μL with an increase of ≥20,000/μL) to at least one prior line of therapy. In some embodiments, the human patient in need thereof has a platelet count of <33,000/μL on two occasions no less than 7 days apart in the 15 days prior to beginning study treatment. In some embodiments, the human patient in need thereof has a history of response (two or more platelet counts ≥50,000/μL with an increase of ≥20,000/μL) to at least one prior line of therapy. In some embodiments, the human patient in need thereof has a platelet count of <33,000/μL on two occasions no less than 7 days apart in the 15 days prior to beginning study treatment and has a history of response (two or more platelet counts ≥50,000/μL with an increase of ≥20,000/μL) to at least one prior line of therapy

In some embodiments, the human patient has a history of response (two or more platelet counts ≥50,000/μL with an increase of ≥20,000/μL) to at least one prior line of therapy. In some embodiments, at least one prior therapy is chosen from a splenectomy, intravenous immunoglobin (IVIG), corticosteroids, anti-D immunoglobulin therapy, and immunosuppressive drugs. In some embodiments, the at least one prior therapy is a splenectomy. In some embodiments, the at least one prior therapy is IVIG. In some embodiments, the at least one prior therapy is corticosteroids. In some embodiments, the at least one prior therapy is anti-D immunoglobulin therapy. In some embodiments, the at least one prior therapy is an immunosuppressive drug.

In some embodiments, the human patient achieves at least one platelet count of at least 20,000/μL above a baseline platelet count, wherein the baseline platelet count is the mean of two platelet counts taken on two occasions no less than 7 days apart in the 15 days prior to beginning treatment and a third count taken on the first day of the study. In some embodiments, the human patient achieves at least two platelet counts of at least 20,000/μL above the baseline platelet count. In some embodiments, the human patient achieves at least two consecutive platelet counts of at least 20,000/μL above the baseline platelet count, for example at least 30,000/μL above the baseline platelet count.

In some embodiments, the human patient achieves at least two consecutive platelet counts of at least 20,000/μL above the baseline platelet count that are separated by at least five days, for example at least seven days. In some embodiments, the human patient achieves at least two consecutive platelet counts of at least 20,000/μL above the baseline platelet count that are separated by at least five days. In some embodiments, the human patient achieves at least two consecutive platelet counts of at least 20,000/μL above the baseline platelet count that are separated by at least seven days.

In some embodiments, the human patient achieves a platelet count of at least 50,000/μL. In some embodiments, the human patient achieves at least two platelet counts of at least 50,000/μL. In some embodiments, the human patient achieves at least two consecutive platelet counts of at least 50,000/μL.

In some embodiments, the human patient achieves at least two consecutive platelet counts of at least 50,000/μL that are separated by at least five days, for example at least seven days. In some embodiments, the human patient achieves at least two consecutive platelet counts of at least 50,000/μL that are separated by at least five days. In some embodiments, the human patient achieves at least two consecutive platelet counts of at least 50,000/μL that are separated by at least seven days.

In some embodiments, the human patient achieves at least one platelet count of at least 50,000/μL within eight days of initiating treatment.

In some embodiments, the human patient does not receive rescue medication during the four weeks prior to the most recent platelet count of at least 50,000/μL.

In some embodiments, the human patient has had ITP for six or less years. In some embodiments, the human patient has had ITP for five or less years. In some embodiments, the human patient has had ITP for four or less years. In some embodiments, the human patient has had ITP for three or less years. In some embodiments, the human patient has had ITP for two or less years. In some embodiments, the human patient has had ITP for less than a year.

In some embodiments, the human patient has a history of taking at least one prior ITP therapy prior to the start of the treatment period. In some embodiments, the human patient has a history of taking at least two prior ITP therapies prior to the start of the treatment period. In some embodiments, the human patient has a history of taking at least three prior ITP therapies prior to the start of the treatment period. In some embodiments, the human patient has a history of taking at least four prior ITP therapies prior to the start of the treatment period. In some embodiments, the human patient has a history of taking at least five prior ITP therapies prior to the start of the treatment period.

In some embodiments, the human patient has platelet counts between 2,000/μL and 33,000/μL on two occasions no less than 7 days apart and within 15 days prior to the start of the treatment period.

In some embodiments, the human patient had a splenectomy prior to the start of the treatment period.

In some embodiments, the human patient has a history of taking at least one TPO-RA prior to the start of the treatment period. In some embodiments, the TPO-RA is selected from recombinant thrombopoietin (rTPO), romiplostim, eltrombopag, and avatrombopag. In some embodiments, the TPO-RA is rTPO. In some embodiments, the TPO-RA is romiplostim. In some embodiments, the TPO-RA is eltrombopag. In some embodiments, the TPO-RA is avatrombopag.

In some embodiments, the human patient has a history of response to at least one prior line of therapy, wherein at least one prior therapy is chosen from a splenectomy, intravenous immunoglobin (IVIG), corticosteroids, anti-D immunoglobulin therapy, and immunosuppressive drugs. In some embodiments, the human patient has a history of response to a splenectomy. In some embodiments, the human patient has a history of response to IVIG. In some embodiments, the human patient has a history of response to corticosteroids. In some embodiments, the human patient has a history of response to anti-D immunoglobulin therapy. In some embodiments, the human patient has a history of response to immunosuppressive drugs.

In some embodiments, the human patient has a history of taking IVIG. In some embodiments, the human patient has a history of taking corticosteroids. In some embodiments, the human patient has a history of taking anti-D immunoglobulin therapy. In some embodiments, the human patient has a history of taking at least one immunosuppressive drug. In some embodiments, the human patient has a history of taking at least one immunosuppressive drug selected from fostamatinib, mycophenolate mofetil (MMF), and cyclosporine. In some embodiments, the human patient has a history of taking fostamatinib. In some embodiments, the human patient has a history of taking mycophenolate mofetil (MMF). In some embodiments, the human patient has a history of taking cyclosporine.

In some embodiments, the response to the prior ITP therapy comprised a platelet count of ≥50,000/μL.

In some embodiments, the human patient has primary ITP. In some embodiments, the human patient has secondary ITP. In some embodiments, the human patient does not have chronic ITP. In some embodiments, the human patient has persistent ITP. In some embodiments, the human patient has chronic ITP. In some embodiments, the human patient has relapsing ITP. In some embodiments, the human patient has refractory ITP.

In some embodiments, the treatment period is at least 8 days. In some embodiments, the treatment period is at least 28 days. In some embodiments, the treatment period is at least 84 days. In some embodiments, the treatment period is at least 169 days.

In some embodiments, the method comprises administering to the human patient 400 mg of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day. In some embodiments, the at least one compound consists of at least one compound chosen from the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof. In some embodiments, the at least one compound consists of at least one compound chosen from the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof. In some embodiments, the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt of the foregoing.

In other embodiments, the method comprises administering to the human patient 400 mg of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile twice a day. In some embodiments, the at least one compound is the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile. In some embodiments, the at least one compound is the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile. In some embodiments, the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.

In some embodiments, the at least one compound is orally administered to the human patient. In some embodiments, the at least one compound is administered to the human patient in the form of at least one tablet. In some embodiments, the at least one compound is administered with water. In some embodiments, the at least one compound is administered with food. In some embodiments, the at least one compound is administered without food.

### Pharmaceutical Compositions:

In some embodiments of the present disclosure, rilzabrutinib is administered as part of a pharmaceutical composition comprising: at least one compound chosen from rilzabrutinib and pharmaceutically acceptable salts thereof; and at least one pharmaceutically acceptable excipient. In some embodiments, the pharmaceutical composition is in the form of at least one tablet.

In some embodiments of the present disclosure, rilzabrutinib is orally administered as part of a pharmaceutical composition comprising: at least one compound chosen from rilzabrutinib and pharmaceutically acceptable salts thereof; and at least one pharmaceutically acceptable excipient. In some embodiments, the pharmaceutical composition is in the form of at least one tablet. In some embodiments, the pharmaceutical composition is in the form of at least one tablet comprising 100 mg or 300 mg of rilzabrutinib. In some embodiments, the pharmaceutical composition is in the form of at least one tablet comprising 100 mg of rilzabrutinib. In some embodiments, the pharmaceutical composition is in the form of at least one tablet comprising 300 mg of rilzabrutinib.

In some embodiments, rilzabrutinib is administered in the form of a film-coated tablet.

In some embodiments of the present disclosure, rilzabrutinib is administered in the form of at least one tablet comprising: at least one compound chosen from rilzabrutinib and pharmaceutically acceptable salts thereof; and at least one pharmaceutically acceptable excipient. In some embodiments, rilzabrutinib is administered in the form of at least one tablet comprising: at least one compound chosen from rilzabrutinib and pharmaceutically acceptable salts thereof; at least one filler; at least one disintegrant; at least one lubricant; and at least one film coating. In some embodiments, the at least one filler is microcrystalline cellulose. In some embodiments, the at least one disintegrant is crospovidone. In some embodiments, the at least one lubricant is sodium stearyl fumarate.

In some embodiments, rilzabrutinib is administered with a glass of water.

In some embodiments, rilzabrutinib is administered with food.

In some embodiments, rilzabrutinib is administered without food.

The proportion and nature of any pharmaceutically acceptable excipient may be determined by the chosen route of administration and standard pharmaceutical practice. Except insofar as any conventional pharmaceutically acceptable excipient is incompatible with rilzabrutinib, such as by producing any undesirable biological effect or otherwise interacting in a deleterious manner with any other component(s) of the pharmaceutically composition, its use is contemplated to be within the scope of this disclosure.

Some non-limiting examples of materials which may serve as pharmaceutically acceptable excipients include: (1) sugars, such as, e.g., lactose, glucose, and sucrose; (2) starches, such as, e.g., corn starch and potato starch; (3) cellulose and its derivatives, such as, e.g., sodium carboxymethyl cellulose, ethyl cellulose, and cellulose acetate; (4) powdered tragacanth; (5) malt; (6) gelatin; (7) talc; (8) excipients, such as, e.g., cocoa butter and suppository waxes; (9) oils, such as, e.g., peanut oil, cottonseed oil, safflower oil, sesame oil, olive oil, corn oil, and soybean oil; (10) glycols, such as, e.g., propylene glycol; (11) polyols, such as, e.g., glycerin, sorbitol, mannitol, and polyethylene glycol; (12) esters, such as, e.g., ethyl oleate and ethyl laurate; (13) agar; (14) buffering agents, such as, e.g., magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York also discloses additional non-limiting examples of pharmaceutically acceptable excipients, as well as known techniques for preparing and using the same.

One skilled in the art can readily select the proper form and route of administration depending upon the disorder or condition to be treated, the stage of the disorder or condition, and other relevant circumstances.

### EXAMPLES

The following example is intended to be illustrative and is not meant in any way to limit the scope of the disclosure.

### Abbreviations

AE Adverse event
ALP Alkaline phosphatase
ALT Alanine aminotransferase
ANC Absolute neutrophil count
aPTT Activated partial thromboplastin time
ASH American Society of Hematology
AST Aspartate aminotransferase
AUC Area under the plasma concentration-time curve
bid/BID Twice daily (morning and evening)
BP Blood pressure
BTK Bruton's Tyrosine Kinase
C Cycle
CA Competent Authority
CBC Complete blood count
CI Confidence Interval
CL/F Apparent total clearance of the drug from
plasma after oral administration
CLL Chronic lymphocytic leukemia
Cmax Maximum observed plasma concentration
CPK Creatine phosphokinase
CRF Case report form
CRO Contract research organization
CS Corticosteroids
CTCAE Common Terminology Criteria for AEs
CYP Cytochrome P450
D Day
DLT Dose-limiting toxicity
DNF Did not fit
EC Ethics Committee
ECG Electrocardiogram
EDC Electronic Data Capture
EQ-5D VAS Euro-QoL 5-Dimension Visual Analog Scale
FSH Follicle Stimulating Hormone
GCP Good Clinical Practice
GFR Glomerular Filtration Rate
H2 Histamine two (receptor)
HCV Hepatitis C Virus
HDPE High-density polyethylene
HIV Human Immunodeficiency Virus
HR Heart rate
HRQOL Health-related quality of life
IB Investigator's Brochure
IBLS ITP Bleeding Scale
ICH International Conference on Harmonization
IDSM Independent Data Safety Monitor
IR Immediate release
IRB Institutional Review Board (Human Research
Ethics Committee)
ITP Immune Thrombocytopenic Purpura
ITP-BAT Idiopathic Thrombocytopenic Purpura
Bleeding Assessment Tool
ITT-E Intent-to-treat exposed
IV Intravenous
IVIG Intravenous immunoglobulin
LPLV Last participant last visit
LTE Long term extension
MAD Multiple ascending dose (trial)
MedDRA Medical Dictionary for Regulatory Activities
NK Natural killer (cell)
NSAID Non-Steroidal Anti-Inflammatory Drug
OTC Over the counter
PE Physical examination
PK Pharmacokinetic
PO Oral
PT/INR Prothrombin Time/International Normalized Ratio
PV Pemphigus vulgaris
PVG Pharmacovigilance
qd/QD Once a day
Q2d/Q2D Every other day
QoL Quality of Life
QT interval corrected for heart rate (Fridiricia
QTcF
Correction)
rTPO Recombinant thrombopoietin
RR Resting Rate
SAE Serious adverse event
SAP Statistical Analytical Plan
SC Subcutaneous
SI Systéme international d'unites (International system of units)
SMC Safety Monitoring Committee
SUSAR Suspected Unexpected Serious Adverse Reaction
TEAE Treatment-Emergent Adverse Event
Tmax Time of observed maximum plasma concentration
TPO Thrombopoietin
TPO-RA Thrombopoietin receptor agonist
t½ Elimination half-life
ULN Upper limit of normal
USUBJID Unique subject identifier
VAS Visual analog scale
WBC White blood cell
WHODD World Health Organization Drug Dictionary

### Example 1: An Adaptive, Open-Label, Dose-Finding, Phase 1/2 Study Investigating the Safety, Pharmacokinetics, and Clinical Activity of Rilzabrutinib, an Oral BTK Inhibitor, in Patients with Relapsed/Refractory Immune Thrombocytopenia (Part A)

An ongoing phase 1/2 clinical trial (NCT03395210) investigating the safety, pharmacokinetics, and clinical activity of rilzabrutinib, an oral BTK inhibitor, in patients with relapsed/refractory immune thrombocytopenia (ITP) began enrolling patients on Mar. 22, 2018. As of Oct. 26, 2022, the estimated primary completion date for the study is March 2023, with an estimated study completion date of March 2024. To date, rilzabrutinib has been well-tolerated in ITP patients, with no reported treatment-related bleeding or thrombotic events. Moreover, positive preliminary results were observed in a highly treatment-resistant and refractory patient population. Of the 60 patients enrolled in the study, 24 patients achieved a primary platelet response. Additionally, of 45 patients who initiated rilzabrutinib at a dose of 400 mg bid, 18 patients achieved the primary platelet response. Moreover, 14 patients who initiated 400 mg bid rilzabrutinib achieved a platelet count of ≥50×10⁹/L by day eight of the study (i.e., an early response).

The key inclusion criteria for the phase 1/2 study were: adults aged 18-80 years old with relapsed/refractory ITP; ITP primary or secondary to other diseases (e.g., systemic lupus erythematosus, chronic lymphocytic leukemia); no other available/approved treatment options; ≥2 platelet counts <30,000/μL at study entry¹; and adequate hematologic, hepatic, and renal function. Key exclusion criteria included: pregnant or lactating women; current drug or alcohol abuse; history of solid organ transplant; and positive screening for HIV, hepatitis B, or hepatitis C. Enrolled patients had low platelet counts, having relapsed on or been refractory to prior therapies with no available and approved therapeutic options and could continue corticosteroids and/or thrombopoietin mimetics during the study. For example, stable concomitant corticosteroid (CS) or thrombopoietin receptor agonist (TPO-RA) treatment was permitted during the study.

The sample size employed in the study was based on clinical considerations with the intention of gaining a sufficiently high confidence level in the study results using normal approximation methods.

### Oversight

The study was designed and conducted to adhere to Good Clinical Practices per the International Conference on Harmonisation E6 requirements and in accordance with the Declaration of Helsinki. The study protocol and informed consent documents were reviewed and approved by the Ethics Committees at each participating institution. All patients provided written informed consent.

The sponsor (Principia/Sanofi) and lead investigator designed the trial in collaboration. Trial conduct was overseen by the sponsor; data were collected by investigators and analyzed by the sponsor. Contents for the first-draft manuscript were led by the first author; medical writing assistance was supported by the sponsor. All authors reviewed, provided feedback on drafts, approved the final manuscript for submission, and vouch for data accuracy and completeness, trial fidelity to the protocol, and complete reporting of adverse events.

### Participants

Eligible immune thrombocytopenia patients were aged 18-80 years (upper age 65 years in Norway/Czech Republic) and platelet counts of <30×10⁹/L on 2 occasions no less ¹ The margin of error for platelet counts can be, for example, ±3,000/μL, depending on the equipment and methods used by the central lab. Therefore, a platelet count of 33,000/μL would be considered a protocol deviation but may not automatically disqualify a patient from the study. than 7 days apart within 15 days prior to study entry. Patients were required to respond to ≥1 prior immune thrombocytopenia therapy (including splenectomy), but at baseline were unable to maintain response to prior/concomitant therapy.

### Inclusion Criteria:

The following inclusion criteria were used to inform the enrollment of patients in the phase 1/2 study, including, e.g., the dose escalation study.
1. Male and female patients, aged 18 to 80 years old (Czech Republic and Norway only: aged 18 to 65 years old)
2. Immune-related ITP (both primary and secondary)
3. Refractory or relapsed patients with no available and approved therapeutic options with a platelet count of count <30,000/μL on two occasions no less than 7 days apart in the 15 days prior to beginning study treatment
4. A history of response (two or more platelet counts ≥50,000/μL with an increase of ≥20,000/μL) to at least one prior line of therapy (with splenectomy being considered a line of therapy)
5. Adequate hematologic, hepatic, and renal function (absolute neutrophil count ≥1.5×10⁹/L, Hgb>9 g/dL, AST/ALT≤1.5×ULN, albumin≥3 g/dL, total bilirubin≤1.5×ULN, estimated GFR>60 (Cockcroft and Gault method) (CID1 pre dose may be checked up to Day −3 prior to CID1)
6. Female patients who are of reproductive potential must agree for the duration of active treatment in the study to use a highly effective means of contraception (hormonal contraception methods that inhibits ovulation, intrauterine device, intrauterine hormone-releasing system, bilateral tubal ligation, vasectomized partner, sexual abstinence). Unless surgically sterile, postmenopausal females should have menopause confirmed by FSH testing.
7. Able to provide written informed consent and agreeable to the schedule of assessment

Additionally, participants could not commence enrollment procedures until all entry criteria had been fulfilled. Where the clinical significance of an abnormal screening test result (lab or any other tests) was uncertain, the test may have been repeated.

### Exclusion Criteria:

The following exclusion criteria were used to inform the enrollment of patients in the phase 1/2 study, including, e.g., the dose escalation study.
1. Pregnant or lactating women
2. ECG findings of QTcF >450 msec (males) or ≥470 msec (females), poorly controlled atrial fibrillation (i.e., symptomatic patients or a ventricular rate above 100 beats/min on ECG), or other clinically significant abnormalities
3. History or current, active malignancy requiring or likely to require chemotherapeutic or surgical treatment during the trial, with the exception of non-melanoma skin cancer
4. Transfusion with blood or blood products or plasmapheresis within 2 weeks before Day 1
5. Change in corticosteroid and/or TPO agonist dose within 2 weeks prior to Day 1 (more than 10% variation from Day 1 daily doses)
6. Use of rescue medications other than corticosteroids or TPO in exclusion #5 in the two weeks before Day 1
7. Immunosuppressant drugs other than corticosteroids—these drugs should be discontinued for at least 14 days before Day 1
8. Treatment with rituximab or splenectomy within the 3 months prior to Day 1
9. Ongoing need for the use of proton pump inhibitor drugs such as omeprazole and esomeprazole (it is acceptable to change patient to H2 receptor blocking drugs prior to Day 1)
10. Concomitant use of known strong-to-moderate inducers or inhibitors of CYP3A, including CYP3A4, within 3 days or 5 half-lives (whichever is longer) of Day 1
11. Use of CYP3A-sensitive substrate drugs with a narrow therapeutic index within 3 days or 5 half-lives (whichever is longer) of study drug dosing including, but not limited to, alfentanil, astemizole, cisapride, cyclosporine, dihydroergotamine, ergotamine, fentanyl, pimozide, quinidine, sirolimus, tacrolimus, or terfenadine
12. Planned or concomitant use of any anticoagulants and platelet aggregation inhibiting drugs such as aspirin, NSAIDs, thienopyridenes (within 14 days of planned dosing through end of follow-up)
13. Has received any investigational drug within the 30 days before receiving the first dose of study medication, or at least 5 times elimination half-life of the drug (whichever is longer); patient should not be using an investigational device at the time of dosing
14. Current drug or alcohol abuse
15. Refractory nausea and vomiting, malabsorption, external biliary shunt, or significant bowel resection that would preclude adequate study drug absorption
16. History of solid organ transplant
17. Positive for screening for HIV, hepatitis B (surface and core antibodies unrelated to vaccination), or hepatitis C (anti-HCV antibody confirmed with Hep C RNA)
18. History of serious infections requiring intravenous therapy within the last 3 months before Day 1
19. Clinically significant cognitive dysfunction (≥Grade 1) or medical history suggestive of increased risk for cognitive dysfunction during the study
20. Live vaccine within 28 days prior to Day 1 or plan to receive one during the study
21. Planned surgery in the time frame of the dosing period
22. Any other clinically significant disease, condition, or medical history that, in the opinion of the Investigator, would interfere with patient safety, study evaluations, and/or study procedures

Additionally, participants must have fulfilled all entry criteria to be enrolled into the study. Participants who failed to meet the entry criteria could be rescreened once at the discretion of the Investigator after informing the study Medical Monitor.

### Trial Design

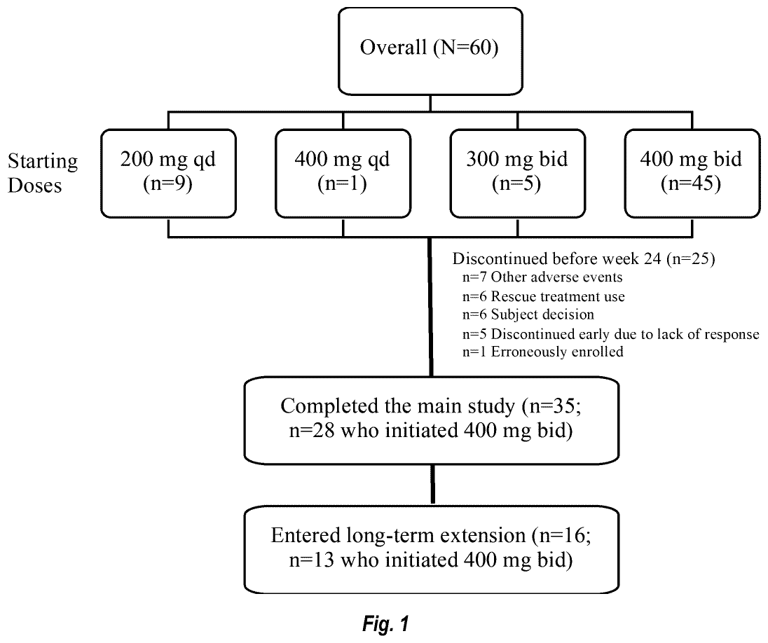

This was a global phase 1-2 adaptive, open-label, dose-finding study of oral rilzabrutinib (Principia Biopharma Inc, a Sanofi Company, South San Francisco, CA) in 8 countries (NCT03395210; EudraCT 2017-004012-19). Rilzabrutinib was administered with intrapatient dose-escalation utilizing a 3+3 design (FIG. 1). Initial doses could be 200 mg once daily (qd), 400 mg qd, 300 mg twice daily (bid; 600 mg/day), or 400 mg bid (800 mg/day; maximum). If no responses were observed at a particular dose level in 3 patients from the sentinel cohort for 28 days, the next higher dose was considered the starting dose. If response was observed at the low dose in 1 of 3 patients, then 3 more patients were added to the cohort. If a patient experienced a platelet response, then the dose was held constant at the next cycle.

Intrapatient dose-escalation was allowed every 28 days per investigator judgment to improve responses. A safety monitoring committee reviewed safety/efficacy data before all patients escalated to a higher dose. The initial study protocol specified a treatment period of 12 weeks; this was subsequently extended to 24-week treatment followed by 4-week safety follow-up. Only stable concomitant glucocorticoid/TPO-RA with ≤10% dosing change within the 2 weeks before rilzabrutinib initiation was allowed throughout treatment (unless rescue criteria were triggered). Responding patients with platelet counts ≥50×10⁹/L, or ≥30×10⁹/L and doubling of baseline for ≥50% of their last 8 weeks of rilzabrutinib could continue in a long-term extension period at 400 mg bid.

Patients discontinued treatment if a dose-limiting toxicity occurred or if rescue criteria were triggered. If the patient required rescue treatment or concomitant ITP drug increases of more than 10% of the day 1 daily dose, the patient received rescue treatment per standard of care, was discontinued from the study, and was considered a nonresponder for subsequent efficacy measures.

### Assessments:

After providing informed written consent, subjects typically completed the following clinical assessments: physical examination; medical history; concomitant medications; weight; height; vital signs; ITP-BAT or IBLS bleeding scale; QOL assessment EQ-5D VAS; online cognitive testing; and safety assessments.

Subjects typically completed the following laboratory and ECG assessments as part of the study:
1. Urinalysis: pH, specific gravity, protein, glucose, ketones, bilirubin, blood, nitrites, urobilinogen and leukocytes measured by dip stick or local requirement
2. Hepatitis B and C, HIV
3. Pregnancy test for women of childbearing potential only. Serum pregnancy tests at screening, urine pregnancy tests at other visits
4. FSH: To confirm postmenopausal status for women who are not surgically sterile and of reproductive potential
5. ABO and Rh blood type
6. Immature Platelet Fraction and Mean Platelet Volume (where available at local lab)
7. Serum chemistry: Aspartate aminotransferase (AST), Alanine aminotransferase (ALT), Total, direct, and indirect bilirubin levels, Alkaline phosphatase (ALP), Albumin, Creatinine, Urea, Total Protein, Sodium, Chloride, Calcium, Phosphate, Potassium, Glucose (random), and creatine phosphokinase (CPK)
8. Hematology (CBC) including differential and reticulocyte counts
9. T/B/NK/monocyte counts by flow cytometry
10. PT/INR PTT
11. TPO levels
12. Hemolysis panel consisting of Coombs test, haptoglobin levels
13. Platelet autoantibody panel (Australia Only: test excluded)
14. PK sampling at various times
15. 12-lead ECG (single and triplicate)

Laboratory assessments could be performed at both central and local laboratories, if required.

Safety assessments included the following: the frequency, severity and relationship of AEs; clinical laboratory test changes; physical examination, ECGs, vital signs, and cognitive function. AEs were coded using the Medical Dictionary for Regulatory Activities version 20.1, and severity graded according to the National Cancer Institute Common Terminology Criteria for AEs (NCI CTCAE), version 4.0. Dose-limiting toxicities (DLTs) during the rilzabrutinib treatment period were predefined in the study protocol. Hematologic DLTs included absolute neutrophil counts (ANCs) of <500/μL for ≥5 days; grade 3 or higher decreased hemoglobin in the absence of a pre-existing grade 2 decreased hemoglobin; febrile neutropenia, with absolute ANC <1000/mm³ and single temperature >38.3° C. or a sustained temperature of ≥38° C. for more than 1 h; or grade ≥3 or higher bleeding event requiring platelet transfusion. Non-hematologic DLTs included any grade ≥3 non-hematologic toxicity per the NCI CTCAE, version 4.0, with the following exceptions: fatigue; laboratory TEAEs, nausea, vomiting, diarrhea, or systemic reactions (such as fever, headache) that returned to baseline or grade 1 within 7 days; or any toxicity that, at the discretion of the investigator warranted withholding the study drug for ≥7 days.

Rescue medication (intravenous immunoglobulin [IVIG], high-dose corticosteroids, platelet infusion or anti-D immunoglobulin infusion) could be used if there was a significant deterioration in the patient's platelet count that in the opinion of the investigator put the patient at significant risk of a safety event. Patients who received rescue medication while on study were discontinued from rilzabrutinib treatment. Response status for patients who received rescue therapy while on study was evaluated based on the platelet counts up until the time of rescue therapy initiation.

In the dose escalation study, patients remained under observation in the clinic for 6 hours after administration of the first dose at the beginning of each new, higher dosing level while having intensive PK sampling performed.

### Dosage Forms:

In the study, rilzabrutinib was administered in the form a film-coated tablet. rilzabrutinib tablets are packaged in white high-density polyethylene (HDPE) bottles with child-resistant induction-sealed caps; these bottles are intended to be stored at 2-8° C. and can be transported without ice at room temperature. Additionally, the bottles can be kept at room temperature conditions for up to 2 weeks.

Each rilzabrutinib film-coated tablet contained either 100 mg or 300 mg of rilzabrutinib drug substance. In addition, the tablet contained Microcrystalline Cellulose (filler), Crospovidone (disintegrant), Sodium Stearyl Fumarate (lubricant), and a non-functional film coating. A 100 mg tablet is a round shape and orange in color. A 300 mg tablet is an oval shape and white in color.

Based on previous studies, food does not appear to impact the extent of rilzabrutinib absorption but reduces the rate (longer average Tₘₐₓ of ˜2.5 hours). Accordingly, rilzabrutinib tablets should be taken with a glass (˜8 oz) of water but may be taken with or without food, i.e., a period of fasting is not required.

### Analysis Populations:

The Screening Population for this study included all participants who provided informed consent and had screening assessments evaluated for study participation.

The Safety Population included all participants who received at least one dose of rilzabrutinib. The Safety Population was used for all safety analyses. For assessment of safety by the IDSM, with regard to dropping a dose level for futility, 3 evaluable patients, defined as compliance of ≥75% of doses for that dose level, were required. During the study, patients were replaced, if necessary, to fulfill this requirement.

The Intent-to-Treat Exposed (ITT-E) Population included all participants who received at least one dose of rilzabrutinib.

The Pharmacokinetic Analysis Population included all participants who received at least one dose of rilzabrutinib and had at least one plasma concentration value that was included in the PK analysis. The Pharmacokinetic Analysis Population was used for all PK analyses.

Participants prematurely discontinued from the study, for reasons other than TEAEs, could be replaced at the discretion of the Sponsor to ensure adequate numbers of evaluable participants.

A patient who withdrew from the study before the planned end of study visit was considered to have withdrawn from the study early. Participants in this study had the right to withdraw at any time for any reason. Additionally, investigators may have withdrawn participants from the study in the event of intercurrent illness, AEs, treatment failure after a prescribed procedure, lack of compliance with the study and/or study procedures, or any other reasons where they felt it was in the best interest of the participant to be terminated from the study.

### Safety and Toxicity Management:

An Independent Data Safety Monitor (IDSM) chosen from expert clinicians in the ITP field provided independent monitoring of the phase 1/2 study. A Safety Monitoring Committee (SMC), comprised of the IDSM as Chairperson, lead Investigator, Study Medical Monitor, and Sponsor's Medical Monitor, also closely supervised the conduct of the study, meeting approximately quarterly and recommending study modification or termination to the Sponsor, based on review of safety and efficacy information. SMC findings that impacted the safety of patients in this study were reported to the local Competent Authority (CA) and IRB/EC.

The IDSM made “sentinel cohort” safety evaluations. The “sentinel patients” for each dose level had their data reviewed by the IDSM, in order to choose the starting dose for additional, new patients. After review, the IDSM could determine that a starting dose for new patients should be dropped for futility (lack of platelet response), increased to the next planned dosing level, kept the same, or reduced. New patients entering the study commenced at the dose level determined by the IDSM based on: (1) if ≥2/3 or ≥2/6 of those sentinel patients have a DLT at any dose level, that level was determined to be the “Maximally Administered Dose” and starting doses (new patients) and continuing doses (patients already on study) were set at lower dosing levels (or study suspended if the current sentinel dose cohort was 200 mg QD); (2) if two or more sustained platelet responses (3 of 4 counts) in the sentinel patients were seen at the current starting dose level the starting dose would not be escalated.

### Clinical Adverse Events

The AE Collection Period began at the time of the first screening/eligibility assessment and will end at the end of the study for each patient. An AE is any untoward medical occurrence in a participant or clinical investigation participant administered a pharmaceutical product and which does not necessarily have to have a causal relationship with the intervention. An AE can therefore be any unfavorable and unintended sign (including an abnormal laboratory finding, for example), symptom, or disease temporally associated with the use of an investigational product, whether or not considered related to the product. Investigators were instructed to record all AEs encountered during the clinical study in detail from the date of participant consent throughout the study follow-up period. Pre-existing conditions that worsen during a study are reported as AEs.

### Adverse Event Relationship to Study Drug

Investigators were instructed to use their knowledge of the study participant, the circumstances surrounding the event, and an evaluation of any potential alternative causes to determine whether an AE may be considered as related to the study drug, indicating “yes” or “no” accordingly. Investigators were asked to consider following information in assessing relatedness: (1) temporal relationship of event onset to the initiation of study drug; (2) course of the event, considering especially the effects of dose reduction, discontinuation of study drug, or reintroduction of study drug (if applicable); (3) known association of the event with the study drug or with similar treatments; (4) known association of the event with the disease under study; (5) presence of risk factors in the study participant or use of concomitant medications known to increase the occurrence of the events; and (6) presence of non-treatment-related factors that are known to be associated with the occurrence of the event.

Investigators were instructed to follow up AEs, especially those for which the severity is Grade 3 or higher, until stabilization or until 4 weeks post last dose (considered as the last follow up), based on the PK profile of the drug.

### Laboratory and ECG Abnormalities

Investigators were instructed to record any treatment-emergent abnormal laboratory or ECG result that is clinically significant, i.e., meeting one or more of the following conditions, as a single diagnosis on the AE page in the CRF. As non-limiting examples, laboratory and ECG abnormalities accompanied by clinical symptoms, leading to a change in study drug (e.g., dose modification, interruption or permanent discontinuation), or requiring a change in concomitant therapy (e.g., addition of, interruption of, discontinuation of, or any other change in a concomitant medication, therapy or treatment) should be recorded as an AE, with any laboratory or ECG result abnormality fulfilling the criteria for a serious adverse event (SAE) reported as such, in addition to being recorded as an AE.

### Adverse Event Intensity Grading

Investigators were instructed to report all clinical AEs encountered during the study. The intensity of AEs is graded based on the NCI CTCAE, Version 4.0 or higher. For any AEs not found in the CTCAE, a description of intensity grading can be found below:
Grade 1: Mild; asymptomatic or mild symptoms; clinical or diagnostic observations only; intervention not indicated.
Grade 2: Moderate; minimal, local or noninvasive intervention indicated; limiting age-appropriate instrumental activities of daily living.
Grade 3: Severe or medically significant but not immediately life-threatening; hospitalization or prolongation of hospitalization indicated; disabling; limiting self-care activities of daily living.

A serious adverse event (SAE) is any experience (clinical AE or abnormal laboratory test) that suggests a significant hazard, contraindication, side effect, or precaution. An SAE must fulfill at least one of the following criteria at any dose level: is fatal (results in the outcome death); is life-threatening; requires in-patient hospitalization or prolongation of existing hospitalization; results in persistent or significant disability/incapacity; is a congenital anomaly/birth defect; or is medically significant or requires intervention to prevent one or other of the outcomes listed above. Investigators were instructed to report life-threatening events or any event with an outcome of death should be reported as an SAE.

### Pregnancy

Any female clinical trial participant who became pregnant during the study was to be instructed to stop taking the study drug and immediately inform the Investigator. Pregnancies occurring up to 90 days after the completion of the study drug were also to be reported to the Investigator.

### Ethical Considerations:

Investigators were tasked with ensuring that the study was conducted in full conformance with the principles of the “Declaration of Helsinki” or with the laws and regulations of the country in which the research is conducted, whichever affords the greater protection to the individual. Additionally, the study adheres to the principles outlined in current “Guideline for Good Clinical Practice” ICH Tripartite Guideline or with local law if it affords greater protection to the participant.

Signed and dated informed consent was obtained from each participant prior to participating in the study after adequate explanation of the aims, methods, objectives and potential hazards of the study. The Investigator or designee must have explained that the participants were completely free to refuse to enter the study or to withdraw from it at any time, for any reason.

### Physical Examination Procedures:

At screening and follow-up visits in the study, a complete physical examination consisted of checking the normality or abnormality of the following body systems: general appearance, skin, eyes, ears, nose, throat, heart, chest/breast, abdomen, neurological system, lymph nodes, spine and extremities (skeletal) and the conduct of an online cognitive testing of learning and memory. An abbreviated physical examination consisted of checking the normality or abnormality of the following body systems: general appearance, skin, abdomen, and cardiorespiratory examination. Height was recorded at screening only. Blood pressure (BP), pulse rate, body temperature and respiratory rate were recorded at specific time points.

Single 12-lead ECG assessments were also obtained at specific time points to confirm eligibility and to ensure real time safety evaluation of the participants in the study. For ECG evaluations, participants should have been in a resting position for at least 10 minutes prior to any measurement. Body position should also have been consistently maintained for each ECG evaluation. In particular, changes in heart rate should have been avoided. There should have been no environmental distractions (TV, radio, conversation) during the pre-ECG rest and the ECG recording time.

Heart rate (HR), QRS duration and respiratory rate (RR), and QT intervals were recorded. Changes of the T-wave and U-wave morphology and overall ECG interpretation were documented. All ECG recordings were performed using a standard high-quality, high-fidelity digital electrocardiograph machine equipped with computer-based interval measurements. For triplicate ECG assessments, at least three interpretable ECG recordings (without artifacts) were collected per time point within a ±10 minute period per time point.

### Laboratory Test Procedures:

Laboratory assessments were performed at a central laboratory, with the provision for occasional local laboratory testing, if required. Laboratory safety tests were collected at specific time points. Additional blood or urine samples may have been taken at an investigator's discretion if the results of any test fell outside the reference ranges, or clinical symptoms necessitated additional testing to monitor participant safety. Where the clinical significance of abnormal lab results was considered uncertain, screening lab tests may have been repeated before Day 1 to confirm eligibility. In the event of unexplained abnormal clinically significant laboratory test values, the tests should have been repeated immediately and followed up until they returned to the normal range, were considered to be clinically stable, and/or an adequate explanation of the abnormality was found.

### Outcomes:

The primary study endpoints were safety and platelet count response. Safety was graded per NCI CTCAE, version 4.0. Platelet response was defined as the proportion of patients achieving ≥2 consecutive platelet counts (separated by ≥5 days) of ≥50×10⁹/L and increased ≥20×10⁹/L from baseline without immune thrombocytopenia rescue medication use in the 4 weeks before the latest elevated platelet count. In cases where a platelet count was missing, the average of non-missing platelet counts was used.

The secondary efficacy endpoints were the percent of weeks with platelet counts ≥50×10⁹/L, the proportion of patients with 4 out of the final 8 platelet counts ≥50×10⁹/L, change from baseline to the average of the post day 1 platelet counts for patients with ≥4 weeks of treatment, number of weeks with platelet counts ≥50×10⁹/L, number of weeks with platelet counts ≥30×10⁹/L, and time to first platelet count ≥50×10⁹/L. Secondary safety endpoints included rescue medication use, proportion of patients with grade ≥2 bleeding events, and bleeding scale scores (using the immune thrombocytopenia-specific bleeding assessment tool (ITP-BAT) (Rodeghiero et al., 2013) and/or immune thrombocytopenia bleeding scale (IBLS)) at the end of the treatment period. Post-hoc analyses are included for responders and nonresponders, as well as subgroup analyses of platelet response that evaluated the potential impact of baseline patient and disease characteristics.

### Statistical Analyses:

The safety population included all patients receiving ≥1 dose of rilzabrutinib. The intent-to-treat population included all patients who enrolled into the study. With an initial sample size of 24 and expected efficacy rate of 40% (i.e., platelet response in 15 evaluable patients), the study could yield 80% confidence that the true proportion would be ≥24% (80% CI±16%) using normal approximation methods. Sample size was later increased to better evaluate outcomes at the highest 400 mg bid dose. Sixty patients were enrolled such that ˜15 patients completed 24 weeks of treatment and >10 patients completed 24 weeks of rilzabrutinib at a 400 mg bid starting dose.

Descriptive statistics and frequency tabulations summarized data per dose and overall. The primary efficacy endpoint and binomial confidence intervals (CI) were analyzed by the Clopper-Pearson method. Kaplan-Meier was used for time-to-event estimates.

### Results

### Patient Characteristics:

The study was initiated on Mar. 22, 2018 (data cutoff May 4, 2021); these results include all 60 patients who enrolled in the study and subsequently had the option to continue in the long-term extension (FIG. 1). Three patients completing the original protocol for 12 weeks of treatment are also included. Patients had a median age of 50 years (range, 19-74) and 57% were female (Table 1). Median baseline platelet counts were 15×10⁹/L (range, 2×10⁹/L-33×10⁹/L). Median duration of immune thrombocytopenia was 6.3 years (range, 0.4-52.5), and patients received a median of 4 prior unique immune thrombocytopenia treatments (range, 1-17). The most common prior therapies were glucocorticoids (92%), TPO-RAs including eltrombopag or romiplostim (58%), IVIG (43%), and rituximab (40%); 25% of patients had a prior splenectomy. Patients receiving glucocorticoids/TPO-RAs at enrollment with inadequate platelet counts (<30×10⁹/L) were allowed to continue stable doses of these medications.

TABLE 1
Characteristics of Patients at Baseline (Safety Population).

| | | | Responders |
|---|---|---|---|
| | | Initiated | who initiated |
| | Overall | 400 mg bid | 400 mg bid |
| Characteristic | (N = 60) | (n = 45) | (n = 18) |
| Median age, | 50 | 49 | 45 |
| y (range) | (19-74) | (19-74) | (22-64) |
| Sex - no. (%) | | | |
| Male | 26 | 18 | 8 |
| | (43) | (40) | (44) |
| Female | 34 | 27 | 10 |
| | (57) | (60) | (56) |
| Median baseline platelet count, | 15 | 15 | 17 |
| ×10⁹/L (range) | (2-33) | (2-33) | (4-29) |
| Median duration of ITP*, | 6.3 | 6.1 | 3.9 |
| years (range) | (0.4-52.5) | (0.4-52.5) | (0.4-18.4) |
| Median number of unique prior | 4 | 4 | 3 |
| ITP therapies^{†} (range) | (1-17) | (1-17) | (1-8) |
| Prior splenectomy^{†} - | 15 | 11 | 3 |
| no. (%) | (25) | (24) | (17) |
| Most common prior ITP | | | |
| therapies^{†} - no. (%) | | | |
| Glucocorticoids | 55 | 42 | Data not |
| | (92) | (93) | provided |
| TPO-RAS | 35 | 24 | |
| | (58) | (53) | |
| IVIG | 26 | 21 | |
| | (43) | (47) | |
| Rituximab | 24 | 22 | |
| | (40) | (49) | |
| Fostamatinib | 8 | 7 | |
| | (13) | (16) | |

*Duration of disease is the difference between the date of first rilzabrutinib dose and the date of initial diagnosis.
^{†}Unique ITP therapies are identified using CMDECOD (the standardized or dictionary-derived text description of CMTRT or CMMODIFY), and splenectomy may be counted as one prior ITP therapy. Stable concomitant glucocorticoid/TPO-RA was defined as ≤10% dosing change within the 2 weeks prior to rilzabrutinib initiation as was allowed per protocol.

### Treatment:

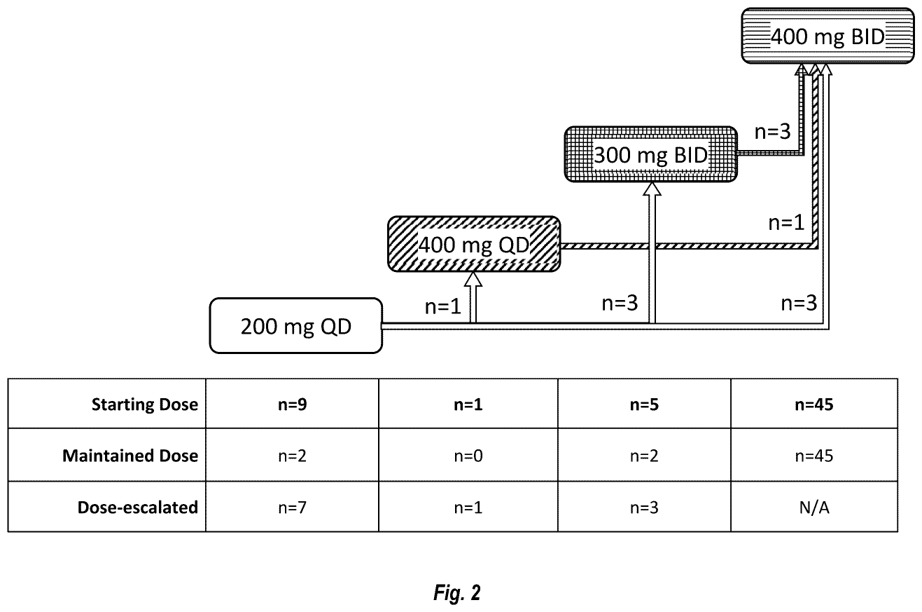

Patients received rilzabrutinib for a median of 167.5 days (range, 4-293). Nine patients initiated treatment at 200 mg qd; of these, 2 patients remained at the 200 mg qd dose, 1 patient dose-escalated to a maximum 400 mg qd, 3 patients to a maximum 300 mg bid (600 mg/day), and 3 patients to a maximum 400 mg bid (800 mg/day; n=3) over the treatment period (FIG. 2). One patient initiating rilzabrutinib at 400 mg qd dose-escalated to a maximum of 400 mg bid. Of five patients initiating 300 mg bid (600 mg/day), three escalated to a maximum dose of 400 mg bid. All 45 patients initiating 400 mg bid remained at that dose with no dose reductions/interruptions due to adverse events and no dose-limiting toxicities.

Overall, 40 patients received concomitant medication (not including rescue medication). The most frequently used were TPO-RAs in 24 patients and glucocorticoids in 23 patients. Out of 24 patients who achieved the primary efficacy endpoint, 4 patients received only concomitant TPO-RAs, 8 only glucocorticoids, 3 both, and 9 had none. Importantly, these patients were considered inadequate responders at baseline.

### Safety:

Rescue medication was used in seven (12%) patients (1 patient at 200 mg qd, 1 at 300 mg bid, 5 at 400 mg bid; Table 2). Use of rescue medication was the cause of study discontinuation (as required by the study protocol) in four patients. Two additional patients received rescue medication but discontinued due to unrelated adverse events. One patient received rescue medication after discontinuing from the study due to lack of response.

TABLE 2
Patients Who Received Rescue Medication(s) and Information on Their Discontinuations Due
to Adverse Events, Lack of Response, and/or Rescue Medication Use During the Study.

| | | Platelet | Discontinuation | | Time From |
|---|---|---|---|---|---|
| | | Count | and/or | | Initial |
| | Baseline | Prior to | Reason | | Treatment |
| Rilzabrutinib | Platelet | Rescue | for Rescue | Rescue | to Rescue |
| Dose* | Count | Medication | Medication | Medication | Use |
| 400 mg bid | 18 × 10⁹/L | 37 × 10⁹/L | Discontinued due | IVIG (iv) | Day 11 |
| | | (C1D8) | to rescue treatment | | |
| | | | use | | |
| 300 mg bid | 3 × 10⁹/L | 14 × 10⁹/L | Discontinued due | Methylprednisolone | Day 29 |
| | | (C2D1) | to rescue treatment | (250 mg qd iv) | |
| | | | use; treatment of | IVIG (70 g iv) | |
| | | | unrelated SAE | TPO-RA | |
| | | | (grade 3 | (eltrombopag 75 | |
| | | | hematoma | g qd oral) | |
| | | | following head | Platelet | |
| | | | contusion due | transfusion | |
| | | | to a fall) | (2 U iv) | |
| 200 mg qd | 24 × 10⁹/L | 4 × 10⁹/L | Discontinued due | Immunoglobulins | Day 36 |
| | | (C2D8) | to rescue treatment | NOS (30000 mg iv) | |
| | | | use | | |
| 400 mg bid | 13 × 10⁹/L | 15 × 10⁹/L | Discontinued due | Romiplostim 4 | Day 80 |
| | | (C3D22) | to rescue treatment | μg/wk sc | |
| | | | use | | |
| 400 mg bid | 16 × 10⁹/L | 3 × 10⁹/L | Treatment of | Methylprednisolone | Day 37 |
| | | (C2D8) | unrelated SAE | (125 and 80 | |
| | | | (grade 2 rectal | mg/d iv) | |
| | | | bleeding) | | |
| 400 mg bid | 17 × 10⁹/L | 8 × 10⁹/L | Treatment of | Methylprednisolone | Day 92 |
| | | (C4D8) | unrelated SAE | (125 mg qd iv) | |
| | | | (grade 4 | Prednisone (80 | |
| | | | thrombocytopenia) | mg po) | |
| | | | | IVIG (130 g qd iv) | |
| 400 mg bid | 21 × 10⁹/L | 16 × 10⁹/L | Lack of response | Anti-D | Day 90 |
| | | (C4D1) | to the study drug | immunoglobulin | |
| | | | | (3750 μg iv) | |

*Rilzabrutinib dose at the time of rescue medication use.

Thirty-one (52%) patients out of the 60 patients who initiated rilzabrutinib at all doses, and 38 (84%) patients out of the 45 patients who initiated rilzabrutinib at 400 mg bid, experienced ≥1 treatment-related, treatment-emergent adverse event; all were grade 1 or 2 and transient (Table 3A and 3B; Table 4).

The most common treatment-related, any-grade treatment-emergent adverse events observed in patients who initiated rilzabrutinib at all doses were diarrhea (32%), nausea (30%), and fatigue (10%) (Table 3A and Table 4). One patient had a treatment-related grade 1 contusion (bleeding) and 1 patient had treatment-related grade 2 erysipelas (infection) that resolved on treatment. No treatment-related grade ≥3 or serious adverse events were observed. There were no signs or symptoms of adverse events typically associated with BTK inhibitors (i.e., neutropenia, treatment-related infections, bleeding, thrombotic events, fungal infections, or atrial fibrillation). In the 45 patients who initiated rilzabrutinib at 400 bid, the most commonly observed treatment-related, treatment-emergent adverse events of any grade were diarrhea (36%), nausea (31%), and fatigue (9%) (Table 3B). There were no treatment-related grade ≥2 bleeding or thrombotic events

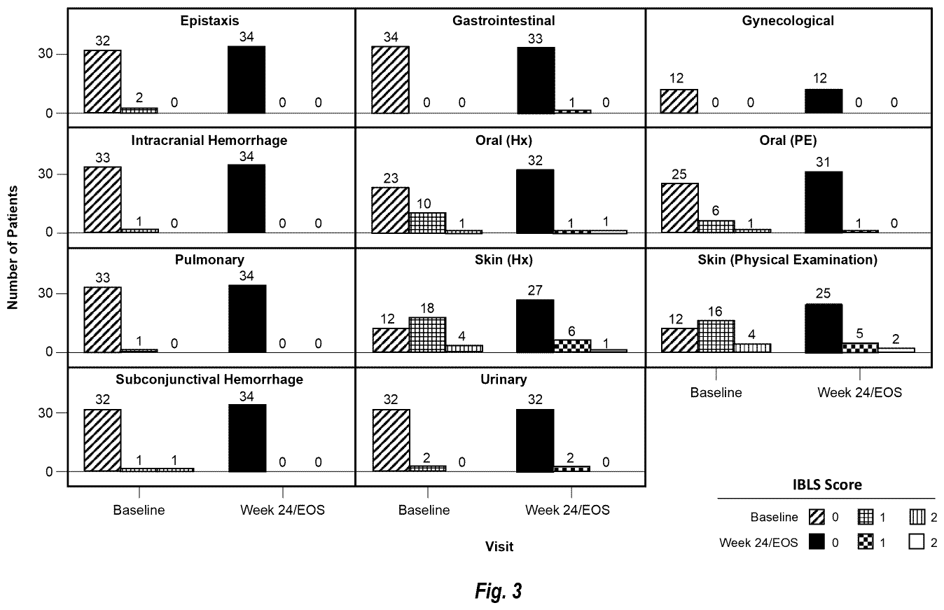

There was 1 patient death unrelated to treatment; this patient discontinued rilzabrutinib 400 mg bid on day 8 due to exacerbation of pre-existing Evans syndrome and died 135 days after initiating the study. The IBLS bleeding scale scores within each domain showed no increase in bleeding from baseline through the end of treatment, but instead improved at skin and oral sites (FIG. 3).

TABLE 3A
Summary of Treatment-Emergent Adverse Events by Grade (Treatment-
Related Events Occurring in ≥5%; Safety Population; N =
60 Patients Who Initiated Rilzabrutinib at All Doses).

| | TEAEs Due to Any Cause | | | | Treatment-Related TEAEs* | | | |
|---|---|---|---|---|---|---|---|---|
| | Any | Grade | Grade | Grade | Any | Grade | Grade | Grade |
| Event | Grade | 1 | 2 | 3/4 | Grade | 1 | 2 | 3/4 |
| | number of patients (percent) | | | | | | | |
| Any TEAE | 48 (80) | 43 (72) | 30 (50) | 8 (13)^{†} | 31 (52) | 27 (45) | 15 (25) | — |
| Diarrhea | 22 (37) | 19 (32) | 3 (5) | — | 19 (32) | 16 (27) | 3 (5) | — |
| Nausea | 21 (35) | 18 (30) | 3 (5) | — | 18 (30) | 16 (27) | 2 (3) | — |
| Fatigue | 12 (20) | 10 (17) | 2 (3) | — | 6 (10) | 5 (8) | 1 (2) | — |
| Abdominal | 6 (10) | 6 (10) | — | — | 4 (7) | 4 (7) | — | — |
| distension | | | | | | | | |
| Vomiting | 4 (7) | 3 (5) | 1 (2) | — | 3 (5) | 2 (3) | 1 (2) | — |

— represents zero events
*Treatment-related TEAEs were all grade 1 or 2; none were grade ≥3.
^{†}Eight patients experienced grade 3/4 TEAEs (multiple may occur in a single patient) due to any cause and unrelated to rilzabrutinib treatment including grade 3 anemia (n = 2); one each with grade 3 abnormal alanine aminotransferase levels, contusion, gastrointestinal hemorrhage, hematoma, immune thrombocytopenia, myelofibrosis, or thrombocytopenia; and one each with grade 4 Evans syndrome or thrombocytopenia.

TABLE 3B
Summary of Treatment-Emergent Adverse Events by Grade
in N = 45 Patients Who Initiated 400 mg bid Rilzabrutinib
(Treatment-Related Events Occurring in ≥5%)

Treatment-Related TEAEs*

| | | |
|---|---|---|
| Event | Grade 1 | Grade 2 |

number of patients (percent)

| | | |
|---|---|---|
| All related TEAEs | 23 (51) | 15 (33) |
| Diarrhea | 13 (29) | 3 (7) |
| Nausea | 12 (27) | 2 (4) |
| Fatigue | 3 (7) | 1 (2) |
| Vomiting | 2 (4) | 1 (2) |

*Treatment-related TEAEs were all grade 1 or 2; none were grade ≥3.

TABLE 4
Summary of Treatment-Emergent Adverse Events by Grade (Occurring in ≥2 Patients
Irrespective of Cause or ≥1 Patient Treatment-Related; Safety Population; N = 60).

| | TEAEs Due to Any Cause | | | | Treatment-Related TEAEs* | | | |
|---|---|---|---|---|---|---|---|---|
| | Any | Grade | Grade | Grade | Any | Grade | Grade | Grade |
| | grade | 1 | 2 | 3/4 | grade | 1 | 2 | 3/4 |
| | number of patients (percent) | | | | | | | |
| Any TEAE | 48 (80) | 43 (72) | 30 (50) | 8 (13)^{†} | 31 (52) | 27 (45) | 15 (25) | 0 (0) |
| Gastrointestinal | | | | | | | | |
| Diarrhea | 22 (37) | 19 (32) | 3 (5) | — | 19 (32) | 16 (27) | 3 (5) | — |
| Nausea | 21 (35) | 18 (30) | 3 (5) | — | 18 (30) | 16 (27) | 2 (3) | — |
| Abdominal | 6 (10) | 6 (10) | — | — | 4 (7) | 4 (7) | — | — |
| distension | | | | | | | | |
| Dyspepsia | 4 (7) | 3 (5) | 1 (2) | — | 1 (2) | 1 (2) | — | — |
| Vomiting | 4 (7) | 3 (5) | 1 (2) | — | 3 (5) | 2 (3) | 1 (2) | — |
| Abdominal pain | 3 (5) | 1 (2) | 2 (3) | — | 2 (3) | — | 2 (3) | — |
| Gingival bleeding | 3 (5) | 3 (5) | — | — | — | — | — | — |
| Upper abdominal | 3 (5) | 2 (3) | 1 (2) | — | 2 (3) | 1 (2) | 1 (2) | — |
| pain | | | | | | | | |
| Abdominal | 1 (2) | 1 (2) | — | — | 1 (2) | 1 (2) | — | — |
| discomfort | | | | | | | | |
| Defecation | 1 (2) | — | 1 (2) | — | 1 (2) | — | 1 (2) | — |
| urgency | | | | | | | | |
| Epigastric | 1 (2) | — | 1 (2) | — | 1 (2) | — | 1 (2) | — |
| discomfort | | | | | | | | |
| Frequent bowel | 1 (2) | — | 1 (2) | — | 1 (2) | — | 1 (2) | — |
| movements | | | | | | | | |
| General disorders | | | | | | | | |
| and administrative | | | | | | | | |
| site conditions | | | | | | | | |
| Fatigue | 12 (20) | 10 (17) | 2 (3) | — | 6 (10) | 5 (8) | 1 (2) | — |
| Peripheral | 3 (5) | 3 (5) | — | — | — | — | — | — |
| swelling | | | | | | | | |
| Edema | 2 (3) | 2 (3) | — | — | 1 (2) | 1 (2) | — | — |
| Malaise | 1 (2) | 1 (2) | — | — | 1 (2) | 1 (2) | — | — |
| Musculoskeletal | | | | | | | | |
| and connective | | | | | | | | |
| tissue disorders | | | | | | | | |
| Back pain | 8 (13) | 7 (12) | 1 (2) | — | 1 (2) | — | 1 (2) | — |
| Arthralgia | 5 (8) | 3 (5) | 2 (3) | — | — | — | — | — |
| Costochondritis | 2 (3) | 2 (3) | — | — | — | — | — | — |
| Muscle spasms | 2 (3) | 2 (3) | — | — | 1 (2) | 1 (2) | — | — |
| Myalgia | 1 (2) | 1 (2) | — | — | 1 (2) | 1 (2) | — | — |
| Infections and | | | | | | | | |
| Infestations^{‡} | | | | | | | | |
| Nasopharyngitis | 3 (5) | 2 (3) | 1 (2) | — | — | — | — | — |
| Upper respiratory | 3 (5) | 2 (3) | 1 (2) | — | — | — | — | — |
| tract infection | | | | | | | | |
| Urinary tract | 2 (3) | 1 (2) | 1 (2) | — | — | — | — | — |
| infection | | | | | | | | |
| Erysipelas | 1 (2) | — | 1 (2) | — | 1 (2) | — | 1 (2) | — |
| Nervous system | | | | | | | | |
| disorders | | | | | | | | |
| Headache | 7 (12) | 7 (12) | — | — | 2 (3) | 2 (3) | — | — |
| Dizziness | 2 (3) | 2 (3) | — | — | — | — | — | — |
| Akathisia | 1 (2) | 1 (2) | — | — | 1 (2) | 1 (2) | — | — |
| Hypoesthesia | 1 (2) | — | 1 (2) | — | 1 (2) | — | 1 (2) | — |
| Respiratory, | | | | | | | | |
| thoracic, and | | | | | | | | |
| mediastinal | | | | | | | | |
| disorders | | | | | | | | |
| Cough | 2 (3) | 2 (3) | — | — | — | — | — | — |
| Dyspnea | 2 (3) | 2 (3) | — | — | — | — | — | — |
| Dyspnea exertional | 2 (3) | 2 (3) | — | — | — | — | — | — |
| Epistaxis | 2 (3) | 2 (3) | — | — | — | — | — | — |
| Nasal congestion | 2 (3) | 1 (2) | 1 (2) | — | — | — | — | — |
| Skin and | | | | | | | | |
| subcutaneous | | | | | | | | |
| tissue | | | | | | | | |
| disorders | | | | | | | | |
| Petechiae | 4 (7) | 4 (7) | — | — | — | — | — | — |
| Rash | 3 (5) | 1 (2) | 2 (3) | — | 1 (2) | 1 (2) | — | — |
| Alopecia | 2 (3) | 2 (3) | — | — | — | — | — | — |
| Purpura | 2 (3) | 2 (3) | — | — | — | — | — | — |
| Pruritus | 2 (3) | 1 (2) | 1 (2) | — | 2 (3) | 1 (2) | 1 (2) | — |
| Maculo-papular | 1 (2) | 1 (2) | — | — | 1 (2) | 1 (2) | — | — |
| rash | | | | | | | | |
| Blood and | | | | | | | | |
| lymphatic | | | | | | | | |
| system | | | | | | | | |
| disorders | | | | | | | | |
| Anemia | 4 (7) | — | 2 (3) | 2 (3) | — | — | — | — |
| Thrombocytopenia | 2 (3) | — | — | 2 (3) | — | — | — | — |
| Metabolism and | | | | | | | | |
| nutrition | | | | | | | | |
| disorders | | | | | | | | |
| Hypophosphatemia | 2 (3) | — | 2 (3) | — | — | — | — | — |
| Decreased appetite | 1 (2) | 1 (2) | — | — | 1 (2) | 1 (2) | — | — |
| Hypokalemia | 1 (2) | 1 (2) | — | — | 1 (2) | 1 (2) | — | — |
| Other | | | | | | | | |
| Contusion | 11 (18) | 7 (12) | 3 (5) | 1 (2) | 1 (2) | 1 (2) | — | — |
| Arthropod bite | 3 (5) | 3 (5) | — | — | — | — | — | — |
| Conjunctival | 5 (8) | 5 (8) | — | — | — | — | — | — |
| hemorrhage | | | | | | | | |
| Heavy menstrual | 2 (3) | 1 (2) | 1 (2) | — | — | — | — | — |
| bleeding | | | | | | | | |
| Abnormal dreams | 1 (2) | 1 (2) | — | — | 1 (2) | 1 (2) | — | — |
| Micturition | 1 (2) | 1 (2) | — | — | 1 (2) | 1 (2) | — | — |
| urgency | | | | | | | | |
| Hot flush | 1 (2) | — | 1 (2) | — | 1 (2) | — | 1 (2) | — |
| Increased white | 1 (2) | — | 1 (2) | — | 1 (2) | — | 1 (2) | — |
| blood cell count | | | | | | | | |

— represents zero events; TEAE, treatment-emergent adverse event.
*Treatment-related TEAEs were all grade 1 or 2; none were grade ≥3.
^{†}Eight patients experienced grade 3/4 TEAEs (multiple may occur in a single patient) due to any cause and unrelated to rilzabrutinib treatment including grade 3 anemia (n = 2); one each with grade 3 abnormal alanine aminotransferase levels, contusion, gastrointestinal hemorrhage, hematoma, immune thrombocytopenia, myelofibrosis, or thrombocytopenia; and one each with grade 4 Evans syndrome or thrombocytopenia.
^{‡}Other infections and infestations not listed in the table that were unrelated to treatment and that occurred in a single patient each included grade 1 candida infection, fungal skin infection, and osteomyelitis; and grade 2 infected skin ulcer and oral herpes.

Seven patients experienced a TEAE that resulted in study drug discontinuation (4 (7%) patients with gastrointestinal (GI) disorders, 2 (4%) with lymphatic system disorders, and 1 (2%) with administrative and site conditions). All were unrelated to rilzabrutinib treatment, with the exception of grade 1 fatigue in 1 patient and grade 2 GI disorders in 2 patients. All TEAEs leading to study drug discontinuation were of grade 1 or 2 severity, with the exception of grade 3 GI hemorrhage, grade 4 thrombocytopenia, and grade 4 Evans syndrome in 1 patient each. Four of the TEAEs leading to study drug discontinuation were reported as serious adverse events (grade 2 rectal hemorrhage, grade 3 GI hemorrhage, grade 4 thrombocytopenia, and grade 4 Evans syndrome); none were related to rilzabrutinib treatment.

### Efficacy:

All 60 patients were evaluable for efficacy for this 24-week study. The primary endpoint (≥2 consecutive platelet counts ≥50×10⁹/L and increased ≥20×10⁹/L from baseline without requiring rescue medication) was achieved in 24 patients (40%; 95 CI, 28%-53%) (Table 5). According to dose level at any time during the study (patients could respond at more than one dose level), 1 of 9 patients (11%) achieved the primary endpoint at 200 mg qd, 2/8 (25%) at 400 mg qd, 4/12 (33%) at 300 mg bid, and 20/52 (38%) at 400 mg bid. In 45 patients who initiated and maintained rilzabrutinib at a dose of 400 mg bid throughout the study, 18 (400) patients achieved the primary platelet response (Table 5; Table 6 efficacy by initial dose).

TABLE 5
Efficacy Outcomes.

| | All Patients | Initiated 400 mg |
|---|---|---|
| | (N = 60) | bid (n = 45) |
| Primary endpoint*, | 24 (40) | 18 (40) |
| n (%) [95% CI] | [28, 53]^{†} | [26, 56] |
| Secondary efficacy endpoints | | |
| Percent of weeks with | | |
| platelet counts | | |
| ≥50 × 10⁹/L, mean (SD) | | |
| All patients | 29 (35) | 28 (36) |
| Responders meeting primary | n = 24 | n = 18 |
| endpoint*^{‡} | 65 (25) | 67 (26) |
| Proportion of patients with >4 | | |
| out of the final 8 platelet | | |
| counts ≥50 × 10⁹/L, n (%) | | |
| [95% CI] | | |
| All patients | 17 (28) | 14 (31) |
| | [17, 41] | [18, 47] |
| Responders meeting primary | n = 24 | n = 18 |
| endpoint*^{‡} | 17 (71) | 14 (78) |
| | [49, 87] | [52, 94] |
| Mean change (SD) from baseline | | |
| to the average of post day | | |
| 1 platelet counts for patients | | |
| with >4 weeks of treatment, ×10⁹/L | | |
| All patients (with >4 weeks treatment) | 29 (40) | 31 (43) |
| Responders meeting primary | n = 24 | n = 18 |
| endpoint*^{‡} | 58 (43) | 64 (48) |
| Number of weeks with platelet | | |
| counts ≥50 × 10⁹/L, median (range) | | |
| All patients | 1 (0-26) | 0 (0-24) |
| Responders meeting primary | n = 24 | n = 18 |
| endpoint*^{‡} | 16 (2-26) | 14 (3-24) |
| Number of weeks with platelet | | |
| counts ≥30 × 10⁹/L, median (range) | | |
| All patients | 5 (0-32) | 5 (0-24) |
| Responders meeting primary | n = 24 | n = 18 |
| endpoint*^{‡} | 21 (3-32) | 21 (7-24) |
| Time to first platelet count ≥50 × | | |
| 10⁹/L, median number of days (range) | | |
| All patients | 11.5 (7-142) | 12.5 (8-142) |
| Responders meeting primary | n = 24 | n = 18 |
| endpoint*^{‡} | 10.5 (7-71) | 11.5 (8-71) |

Note:
Efficacy endpoints by initial dose level of rilzabrutinib for the 24-week study (and prior to the long-term extension) are reported in Table 6.
CI, confidence interval (based on Wilson Score method). The widths of the confidence intervals have not been adjusted for multiplicity and should not be used to definitively infer efficacy.
*The primary endpoint for platelet response was defined as ≥2 consecutive platelet counts, separated by at least 5 days, of ≥50 × 10⁹/L and increased ≥20 × 10⁹/L from baseline without requiring rescue medication in the 4 weeks prior to the latest elevated platelet count.
^{†}Included achievement of primary endpoint at rilzabrutinib doses of 200 mg qd (n = 1), 400 mg qd (n = 2), 300 mg bid (n = 4), and 400 mg bid (n = 20) at any time; primary endpoint may have been reached at different doses in the same patient.
^{‡}Post-hoc analyses of responding patients.

TABLE 6
Efficacy Outcomes by Initial Dose of Rilzabrutinib.

| | 200 mg | 400 mg | 300 mg | 400 mg | All |
|---|---|---|---|---|---|
| Initial Dose of | qd | qd | bid | bid | Patients |
| Rilzabrutinib | (n = 9) | (n = 1) | (n = 5) | (n = 45) | (N = 60) |
| Primary endpoint*, | 4 (44) | 0 (0) | 2 (40) | 18 (40) | 24 (40) |
| n (%) [95% CI] | [14, 79] | [0, 98] | [5, 85] | [26, 56] | [28, 53] |
| Secondary efficacy | | | | | |
| endpoints | | | | | |
| Percent of weeks with | 28 | 0 | 40 | 28 | 29 |
| platelet counts ≥50 × | (29) | (NA) | (41) | (36) | (35) |
| 10⁹/L, mean (SD) | | | | | |
| Proportion of patients | 1 (11) | 0 (0) | 2 (40) | 14 (31) | 17 (28) |
| with ≥4 out of the final | [0.3, 48] | [0, 98] | [5, 85] | [18, 47] | [17, 41] |
| 8 platelet counts ≥50 × | | | | | |
| 10⁹/L, n (%) [95% CI] | | | | | |
| Mean change (SD) | n = 8 | n = 1 | n = 4 | n = 41 | n = 54 |
| from baseline to the | 19 (24) | −0.4 (NA) | 30 (27) | 31 (43) | 29 (40) |
| average of post day 1 | | | | | |
| platelet counts for | | | | | |
| patients with >4 weeks | | | | | |
| of treatment, ×10⁹/L | | | | | |
| Number of weeks with | 2.5 | 0 | 5 | 0 | 1 |
| platelet counts ≥50 × | (0-20) | (0-0) | (0-26) | (0-24) | (0-26) |
| 10⁹/L, median | | | | | |
| (range) | | | | | |
| Number of weeks with | 4 | 0 | 15 | 5 | 5 |
| platelet counts ≥30 × | (0-28) | (0-0) | (0-32) | (0-24) | (0-32) |
| 10⁹/L, median | | | | | |
| (range) | | | | | |
| Time to first platelet | 8 | — | 8 | 12.5 | 11.5 |
| count ≥50 × 10⁹/L, | (8-63) | | (7-8) | (8-142) | (7-142) |
| median number of | | | | | |
| days (range) | | | | | |

CI, confidence interval (based on Wilson Score method); NA, not applicable; NE, not evaluated. The widths of the confidence intervals have not been adjusted for multiplicity and should not be used to definitively infer efficacy.
*The primary endpoint for platelet response was defined as ≥2 consecutive platelet counts, separated by at least 5 days, of ≥50 × 10⁹/L and increased ≥20 × 10⁹/L from baseline without requiring rescue medication in the 4 weeks prior to the latest elevated platelet count.

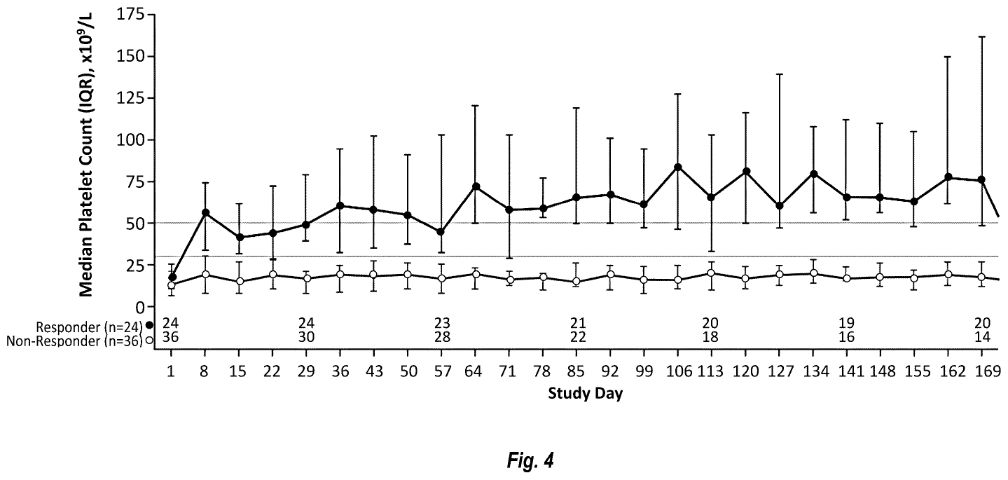

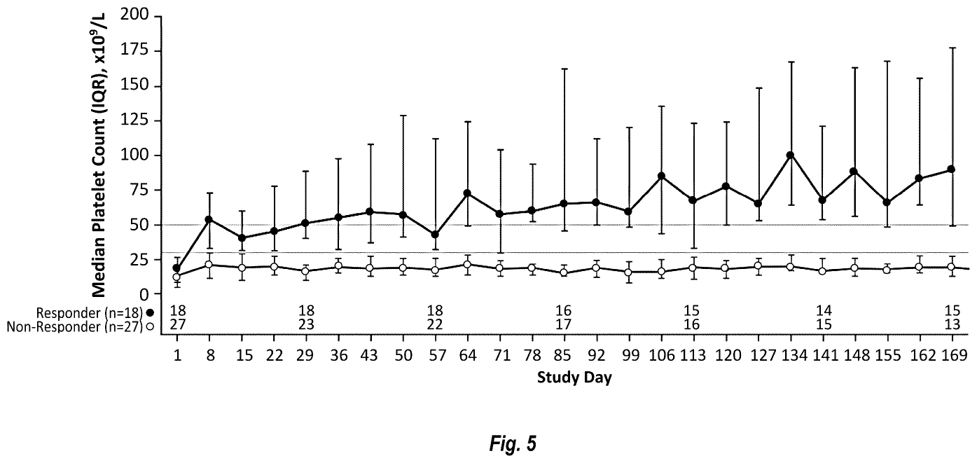

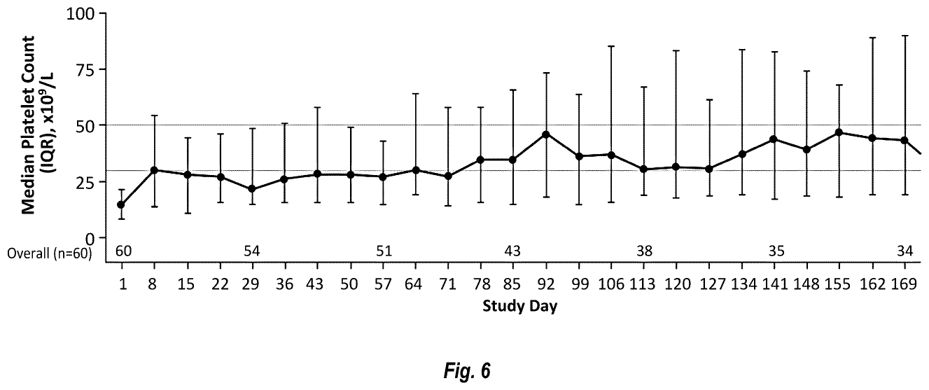

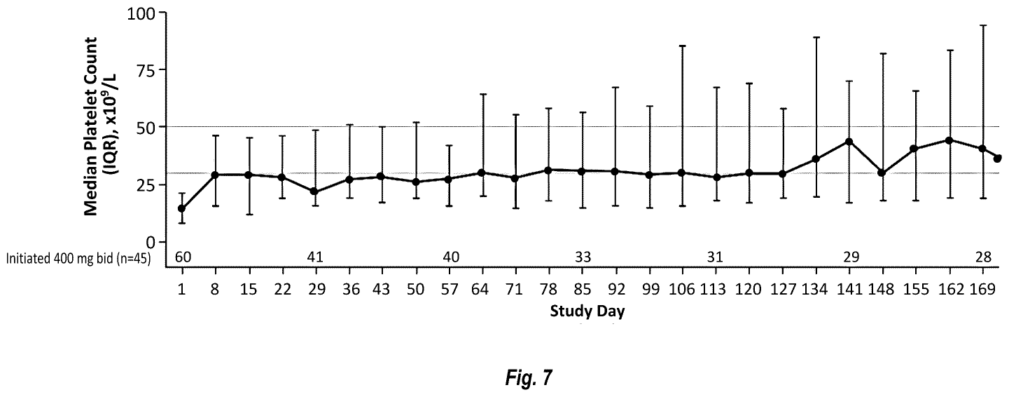

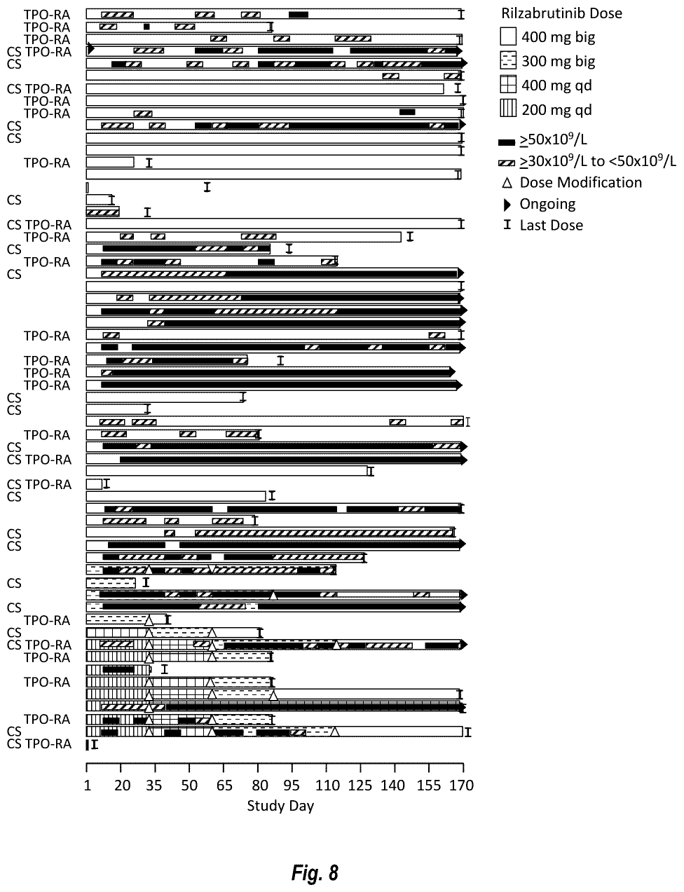

The mean percent of weeks with platelet counts ≥50×10⁹/L was 29% overall; post-hoc analyses showed that the mean percent of weeks was 65% for responder patients meeting the primary endpoint. Based on a safety profile that was low-grade and consistent efficacy observed at the highest evaluated dose, the minimum effective dose for rilzabrutinib was identified as 400 mg bid. No escalations beyond 400 mg bid were tested. The proportion of patients with ≥4 of final 8 platelet counts ≥50×10⁹/L was 17 (28%) overall and 14 (31%) for patients initiating 400 mg bid (Table 5). Mean changes from baseline to the average of post-day 1 platelet counts in patients completing >4 weeks of treatment were 29×10⁹/L (SD, 40×10⁹/L) for all patients and 31×10⁹/L (SD, 43×10⁹/L) for patients initiating 400 mg bid. Platelet counts of ≥50×10⁹/L and ≥30×10⁹/L were maintained for median 1 and 5 weeks, respectively, in the overall patient population. When evaluating responding patients overall (n=24) and those initiating 400 mg bid (n=18) who met the primary efficacy platelet counts, these respective groups maintained platelet counts of ≥50×10⁹/L for a median of 16 and 14 weeks, as well as platelet counts of ≥30×10⁹/L for a median of 21 weeks for both groups (FIGS. 4 and 5). The median time to first platelet count ≥50×10⁹/L was 11.5 days for all patients and 12.5 days for those initiating 400 mg bid (FIGS. 6-8, Table 5, Table 6).

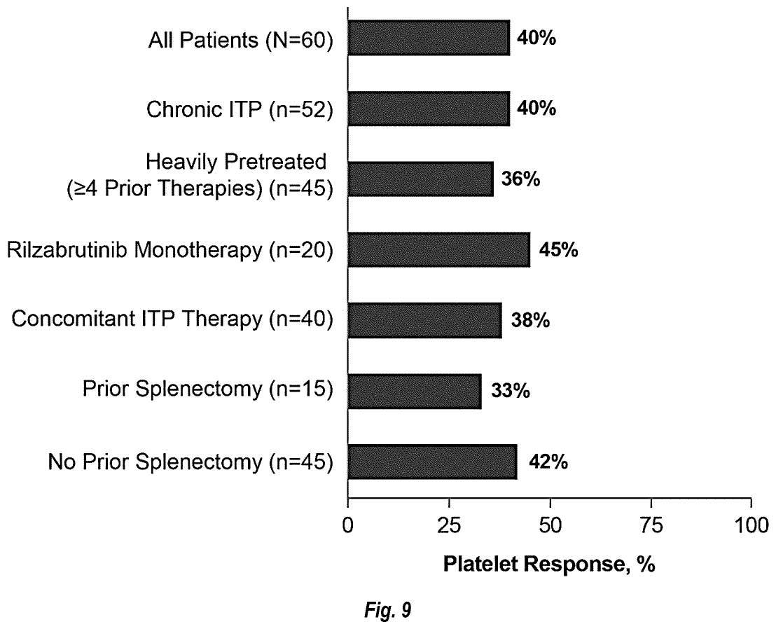

Primary platelet responses overall were comparable across subgroups of patients who had chronic immune thrombocytopenia (duration for ≥12 months) and persistent immune thrombocytopenia (duration of 3-12 months), were heavily pretreated with ≥4 prior immune thrombocytopenia therapies, had received either rilzabrutinib monotherapy, concomitant immune thrombocytopenia therapy, or prior splenectomy (FIG. 9).

This clinical study demonstrated that BTK inhibition with rilzabrutinib is effective at suppressing immune-mediated platelet destruction in immune thrombocytopenia, thus providing a novel mechanism for targeting underlying immune thrombocytopenia pathology. Patients had experienced immune thrombocytopenia for a median duration of 6.3 years with a median of four unique prior therapies. Notably, eligibility was based on an inability to maintain an adequate response to prior/concomitant therapies (including splenectomy) and ongoing need for treatment. Oral rilzabrutinib was associated with only low-grade toxicity across all doses, in alignment with early clinical studies in healthy volunteers (Smith P F et al., 2017) and projected doses from preclinical studies (Langrish C L et al., 2021). There was no evidence of infections, thrombotic events, cardiac arrhythmias, liver toxicity, or increased bleeding typically associated with BTK inhibitors and/or TPO-RAs though follow up time was limited (Aguilar C, 2018; Shatzel J J et al., 2017; Ghanima W et al., 2018; von Hundelshausen P and Seiss W, 2021). In addition, no patient developed platelet counts >400×10⁹/L. Rilzabrutinib's safety profile was consistent with prior phase 1 healthy volunteer results (Smith P F et al., 2017).

Rilzabrutinib led to rapid platelet responses and clinically significant platelet responses (≥50×10⁹/L) in 40% of rilzabrutinib-treated patients. Important treatment factors for immune thrombocytopenia patients are attaining rapid improvement and maintaining durable responses over time. Median time to first platelet count ≥50×10⁹/L was rapid at 11.5 days, which was maintained in responders for a median of 72% of weeks in the 24-week treatment period. Importantly, platelet responses were consistent across baseline subgroups.

No standard treatment recommendations currently exist for immune thrombocytopenia patients with multiple relapses. Treatment/study comparisons are further complicated by a range of timepoints and platelet response endpoints. Recent ASH treatment guidelines for immune thrombocytopenia lasting ≥3 months recommend TPO-RAs, rituximab, or splenectomy (Neunert et al., 2019). Immunomodulators for relapsed immune thrombocytopenia include spleen tyrosine kinase inhibitor fostamatinib (Tavalisse (fostamatinib) prescribing information, 2020) and anti-CD20 antibody rituximab (Ghanima W et al., 2015; Deshayes S et al., 2019; Tjonnfjord E et al., 2020). Although durable remissions are high with splenectomy (˜60-70% of patients), it is generally viewed less favorably due to potential surgical complications, higher risk for infections/thromboembolic events, and lifelong increased risk of sepsis with capsuled microorganisms (Cooper N and Ghanima W, 2019; Kojouri K et al., 2004). Treatment choice is generally individualized based on patient age/characteristics, comorbidities, disease duration, bleeding frequency, treatment availability, and patient preferences (Neunert et al., 2019). Despite significant advances in immune thrombocytopenia therapeutic options over the last 15 years, a subset of patients remains refractory to existing therapies and durable remission remains elusive. Because the patients here were heavily pretreated and many were viewed by investigators as having limited treatment options, inhibition of BTK by rilzabrutinib provided a novel therapeutic approach by targeting immune-mediated platelet destruction and impaired platelet production.

This phase 1-2, dose-finding clinical study established a role for BTK inhibition in immune thrombocytopenia treatment. Overall, oral rilzabrutinib demonstrated rapid and durable clinical activity in up to half of immune thrombocytopenia patients following multiple prior therapies and had a low-grade safety profile. The rilzabrutinib 400 mg bid dose was shown as safe and effective.

### Example 2: An Adaptive, Open-Label, Dose-Finding, Phase 1/2 Study Investigating the Safety, Pharmacokinetics, and Clinical Activity of Rilzabrutinib, an Oral BTK Inhibitor, in Patients with Relapsed/Refractory Immune Thrombocytopenia (Part B)

Part B of the open-label, phase 1/2 study continued to evaluate the safety and efficacy of 400 mg BID rilzabrutinib in patients with relapsed ITP. Adult patients (aged 18-80 years) with ≥2 baseline platelet counts <30×10⁹/L no less than 7 days apart in the 15 days before the first dose. Eligible patients were required to have a past response (achievement of platelet count ≥50×10⁹/L) to IVIG/anti-D or CS that was not sustained, and to have failed ≥1 other non-IVIG, non-CS ITP therapy. Stable doses of concomitant CS/TPO-RA were allowed with rilzabrutinib. The primary endpoints were safety and durable platelet response (defined as platelet counts ≥50×10⁹/L on ≥8 of the last 12 weeks of rilzabrutinib without rescue medication). Consistent with the results from Part A, rilzabrutinib demonstrated rapid, stable, and durable platelet responses in patients with relapsed ITP, with a favorable safety profile in part B.

Change from baseline in the ITP Bleeding Scale (IBLS) score (0 none to 2 marked bleeding) was a secondary endpoint to assess bleeding across 9 anatomical sites (8 anatomical sites for male/postmenopausal women). An exploratory health-related quality of life (HRQOL) endpoint evaluated the EuroQol-5 Dimensions 5-Level (EQ-5D-5L)+Visual Analog Scale (EQ-VAS) and ITP Patient Assessment Questionnaire™ (ITP-PAQ) scores (0 worst to 100 best QOL). Overall, patients treated with rilzabrutinib exhibited a durable platelet responses, high compliance, and improvements on HRQOL measures in difficult to treat patients with relapsed ITP. There was no evidence of increased bleeding with rilzabrutinib, with scores improving at the skin site. Clinically meaningful improvements in HRQOL were observed in multiple individual and overall HRQOL health domains following rilzabrutinib.

### Example 3: Clinical Predictors of Response to Rilzabrutinib Therapy in Patients with Immune Thrombocytopenia: Exploratory Analysis of a Phase 1/2 Study

A total of 11 baseline clinical variables and pharmacokinetics (PK) exposure at steady-state were selected as potential predictors of platelet response among 45 patients who initiated 400 mg rilzabrutinib twice daily (BID) (See Example 1). Platelet response was defined as ≥2 consecutive platelet counts of ≥50×10⁹/L, separated by ≥5 days, and an increase from baseline of ≥20×10⁹/L without the use of rescue medication during the 4 weeks before the latest elevated platelet count, and early response was defined as having platelet counts ≥50×10⁹/L by day 8. Clinical predictors were selected based on clinical considerations anticipated to be associated with platelet response and evaluated by univariate logistic regression models. For continuous variables, the t test was used to test for differences, whereas for categorical variables, the Fisher exact test was used. PK exposure at steady-state was estimated based on post hoc analysis from a previously established population PK model.

### Results

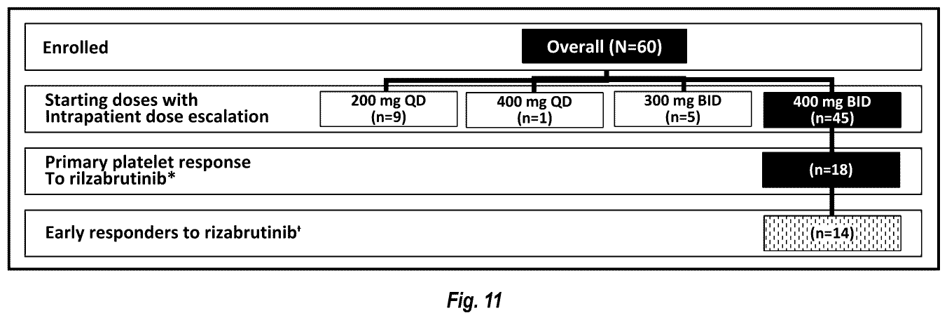

Of the 45 patients who initiated 400 mg BID rilzabrutinib, the median age was 49 years (Table 1). Patients had a median ITP duration of 6.1 years, a median platelet count of 15×10⁹/L, and a median of four prior ITP therapies. 24% of patients had received a splenectomy prior to initiating treatment. 18 patients achieved the primary platelet response, of whom 14 were early responders (FIG. 11).

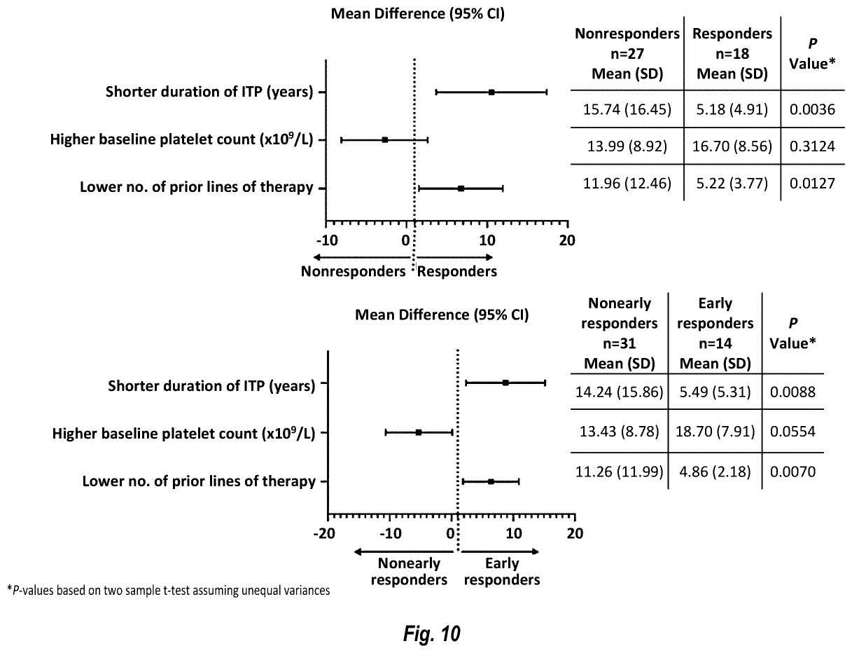

Significant predictors of response to rilzabrutinib, based on the univariate logistic regression models, were shorter duration of ITP (P=0.04) and no prior use of thrombopoietin receptor agonists (TPO-RA; P=0.03) or rituximab (P=0.02; Table 7). No prior rituximab therapy was also a significant predictor in early responders (P=0.02; Table 7). Conversely, the use of concomitant ITP medication with TPO-RA or corticosteroids was not associated with predicting either response or early response to rilzabrutinib treatment. Similar results were noted upon assessment of continuous variables by mean difference. Compared with their counterparts, responders had a significantly shorter mean duration of ITP (P<0.01) and lower number of prior lines of therapy (P=0.01) as did the early responders (P<0.01 for both; FIG. 10). Across categorical variables assessed by comparing proportions of patients, significant associations with being a responder vs nonresponder were seen with no prior use of TPO-RA (P=0.05) and lack of prior rituximab therapy (P=0.03). Responders accounted for 29% vs 64% of patients with vs without prior TPO-RA and 23% vs 57% of those with vs without prior rituximab. No prior rituximab was also significantly associated with early response (P=0.02) to rilzabrutinib, with 48% of these patients being early responders vs 14% of those who received prior rituximab. None of the PK exposure metrics significantly impacted response to rilzabrutinib.

TABLE 7
Individual Logistic Regression Model Results.

| | | Odds Ratio | 95% CI | P Value |
|---|---|---|---|---|
| Responders (n = 18) | | | | |
| Baseline platelet count | | 1.037 | 0.967-1.112 | .3084 |
| (increase by 1 × 10⁹/L) | | | | |
| Duration of ITP | | 0.915 | 0.842-0.994 | .0365 |
| (increase by 1 year) | | | | |
| ITP diagnosis group | | Model | | |
| (primary ITP) | | D.N.F. | | |
| Number of prior | | 0.863 | 0.740-1.006 | .0597 |
| lines of therapy | | | | |
| (increase by 1) | | | | |
| Prior | No prior | 2.105 | 0.475-9.338 | .3273 |
| treatment | splenectomy | | | |
| | No prior | 4.399 | 1.151-16.805 | .0303 |
| | TPO-RA | | | |
| | No prior | Model | | |
| | corticosteroids | D.N.F. | | |
| | No prior | 4.420 | 1.212-16.118 | .0244 |
| | rituximab | | | |
| Concomitant | No concomitant | 1.600 | 0.463-5.527 | .4574 |
| medication | TPO-RA | | | |
| | No concomitant | 0.625 | 0.183-2.131 | .4527 |
| | CS | | | |
| | No concomitant | Model | | |
| | rituximab | D.N.F. | | |
| Early Responders (n = 14) | | | | |
| Baseline platelet count | | 1.076 | 0.995-1.165 | .0679 |
| (increase by 1 × 10⁹/L) | | | | |
| Duration of ITP | | 0.930 | 0.857-1.010 | .0848 |
| (increase by 1 year) | | | | |
| ITP diagnosis group | | Model | | |
| (primary ITP) | | D.N.F. | | |
| Number of prior | | 0.843 | 0.698-1.017 | .0750 |
| lines of therapy | | | | |
| (increase by 1) | | | | |
| Prior | No prior | 2.455 | 0.455-13.248 | .2965 |
| treatment | splenectomy | | | |
| | No prior | 3.429 | 0.894-13.147 | .0724 |
| | TPO-RA | | | |
| | No prior | Model | | |
| | corticosteroids | D.N.F. | | |
| | No prior | 5.805 | 1.339-25.169 | .0188 |
| | rituximab | | | |
| Concomitant | No concomitant | 2.059 | 0.529-8.008 | .2974 |
| medication | TPO-RA | | | |
| | No concomitant | 0.733 | 0.202-2.662 | .6372 |
| | CS | | | |
| | No concomitant | Model | | |
| | rituximab | D.N.F. | | |

P value < 0.05 is considered significant.

In heavily pretreated patients with ITP, the strongest predictors of response appeared to be short duration of ITP and no history of rituximab therapy, suggesting that patients who are more treatment naïve may be more likely to respond to rilzabrutinib therapy than those receiving prior ITP medication.

### Embodiments

Non-limiting embodiments of the disclosure include:
1. A method for treating immune thrombocytopenia (ITP) in a human patient in need thereof who has not received prior treatment with a thrombopoietin receptor agonist (TPO-RA) or rituximab comprising administering to the human patient who has not received prior treatment with a TPO-RA or rituximab a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day for a treatment period, wherein the human patient in need thereof has at least one characteristic chosen from:
   a. a platelet count of count <33,000/μL on two occasions no less than 7 days apart in the 15 days prior to beginning study treatment; and
   b. a history of response (two or more platelet counts ≥50,000/μL with an increase of ≥20,000/μL) to at least one prior line of therapy.
2. A method for treating immune thrombocytopenia (ITP) in a human patient in need thereof who has not received prior treatment with a thrombopoietin receptor agonist (TPO-RA) comprising administering to the human patient who has not received prior treatment with a TPO-RA a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day for a treatment period, wherein the human patient in need thereof has at least one characteristic chosen from:
   a. a platelet count of <33,000/μL on two occasions no less than 7 days apart in the 15 days prior to beginning study treatment; and
   b. a history of response (two or more platelet counts ≥50,000/μL with an increase of ≥20,000/μL) to at least one prior line of therapy.
3. A method for treating immune thrombocytopenia (ITP) in a human patient in need thereof who has not received prior treatment with rituximab comprising administering to the human patient who has not received prior treatment with rituximab a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day for a treatment period, wherein the human patient in need thereof has at least one characteristic chosen from:
   a. a platelet count of <33,000/μL on two occasions no less than 7 days apart in the 15 days prior to beginning study treatment; and
   b. a history of response (two or more platelet counts ≥50,000/μL with an increase of ≥20,000/μL) to at least one prior line of therapy.
4. The method according to any one of Embodiments 1 or 2, wherein the TPO-RAs are chosen from: recombinant thrombopoietin (rTPO), romiplostim, eltrombopag, and avatrombopag.
5. The method according to any one of Embodiments 1-2 or 4, wherein the patient has not received prior treatment with rituximab.
6. The method according to any one of Embodiments 1-5, wherein the at least one prior line of therapy, wherein at least one prior therapy is chosen from a splenectomy, intravenous immunoglobin (IVIG), corticosteroids, anti-D immunoglobulin therapy, and immunosuppressive drugs.
7. The method according to any one of Embodiments 1-6, wherein the patient achieves at least one platelet count of at least 20,000/μL above a baseline platelet count, wherein the baseline platelet count is the mean of two platelet counts taken on two occasions no less than 7 days apart in the 15 days prior to beginning treatment and a third count taken on the first day of the study.
8. The method according to any one of Embodiments 1-7, wherein the patient achieves at least two platelet counts of at least 20,000/μL above the baseline platelet count.
9. The method according to Embodiment 8, wherein the patient achieves at least two consecutive platelet counts of at least 20,000/μL above the baseline platelet count.
10. The method according to any one of Embodiments 8 or 9, wherein at least two consecutive platelet counts of at least 20,000/μL above the baseline platelet count are separated by at least five days, for example at least seven days.
11. The method according to any one of Embodiments 1-10, wherein the patient achieves a platelet count of at least 50,000/μL.
12. The method according to any one of Embodiments 1-11, wherein the patient achieves at least two platelet counts of at least 50,000/μL.
13. The method according to Embodiment 12, wherein the patient achieves at least two consecutive platelet counts of at least 50,000/μL.
14. The method according to any one of Embodiments 12 or 13, wherein the at least two consecutive platelet counts of at least 50,000/μL are separated by at least five days, for example at least seven days.
15. The method according to any one of Embodiments 1-14, wherein the human patient achieves at least one platelet count of at least 50,000/μL within eight days of initiating treatment.
16. The method according to any one of Embodiments 11-15 wherein the human patient does not receive rescue medication during the four weeks prior to the most recent platelet count of at least 50,000/μL.
17. The method according to any one of Embodiments 1-16, wherein the human patient has had ITP for six or less years.
18. The method according to any one of Embodiments 1-17, wherein the human patient has a history of taking at least one prior ITP therapy prior to the start of the treatment period.
19. The method according to any one of Embodiments 1-18, wherein the human patient has a history of taking at least two prior ITP therapies prior to the start of the treatment period.
20. The method according to any one of Embodiments 1-19, wherein the human patient has a history of taking at least three prior ITP therapies prior to the start of the treatment period.
21. The method according to any one of Embodiments 1-20, wherein the human patient has a history of taking at least four prior ITP therapies prior to the start of the treatment period.
22. The method according to any one of Embodiments 1-21, wherein the human patient has a history of taking at least five prior ITP therapies prior to the start of the treatment period.
23. The method according to any one of Embodiments 1-22, wherein the human patient has platelet counts between 2,000/μL and 33,000/μL on two occasions no less than 7 days apart and within 15 days prior to the start of the treatment period.
24. The method according to any one of Embodiments 1-23, wherein the human patient had a splenectomy prior to the start of the treatment period.
25. The method according to any one of Embodiments 2 or 4-24, wherein the human patient has a history of taking rituximab prior to the start of the treatment period.
26. The method according to any one of Embodiments 3-25, wherein the human patient has a history of taking at least one TPO-RA prior to the start of the treatment period.
27. The method according to Embodiment 26, wherein the at least one TPO-RA is selected from rTPO, romiplostim, eltrombopag, and avatrombopag.
28. The method according to any one of Embodiments 1-27, wherein the patient has a history of response to at least one prior line of therapy, wherein at least one prior therapy is chosen from a splenectomy, intravenous immunoglobin (IVIG), corticosteroids, anti-D immunoglobulin therapy, and immunosuppressive drugs.
29. The method according to any one of Embodiments 1-28, wherein the human patient has a history of taking intravenous immunoglobin (IVIG) prior to the start of the treatment period.
30. The method according to any one of Embodiments 1-28, wherein the human patient has a history of taking corticosteroids prior to the start of the treatment period.
31. The method according to any one of Embodiments 1-28, wherein the human patient has a history of taking anti-D immunoglobulin therapy prior to the start of the treatment period.
32. The method according to any one of Embodiments 1-28, wherein the human patient has a history of taking at least one immunosuppressive drug prior to the start of the treatment period.
33. The method according to Embodiment 32, wherein the at least one immunosuppressive drug is selected from fostamatinib, mycophenolate mofetil (MMF), and cyclosporine.
34. The method according to any one of Embodiments 28-33, wherein the response to the prior ITP therapy comprised a platelet count of ≥50,000/μL.
35. The method according to any one of Embodiments 1-34, wherein the human patient has primary ITP.
36. The method according to any one of Embodiments 1-34, wherein the human patient has secondary ITP.
37. The method according to any one of Embodiments 1-34, wherein the human patient does not have chronic ITP.
38. The method according to any one of Embodiments 1-34, wherein the human patient has persistent ITP.
39. The method according to any one of Embodiments 1-34, wherein the human patient has chronic ITP.
40. The method according to any one of Embodiments 1-34, wherein the human patient has relapsing ITP.
41. The method according to any one of Embodiments 1-34, wherein the human patient has refractory ITP.
42. The method according to any one of Embodiments 1-41, wherein the treatment period is at least 8 days.
43. The method according to any one of Embodiments 1-42, wherein the treatment period is at least 28 days.
44. The method according to any one of Embodiments 1-43, wherein the treatment period is at least 84 days.
45. The method according to any one of Embodiments 1-44, wherein the treatment period is at least 169 days.
46. The method according to any one of Embodiments 1-45, comprising administering to the human patient 400 mg of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day.
47. The method according to any one of Embodiments 1-46, wherein the at least one compound consists of at least one compound chosen from the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof.
48. The method according to any one of Embodiments 1-46, wherein the at least one compound consists of at least one compound chosen from the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof.
49. The method according to any one of Embodiments 1-46, wherein the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt of the foregoing.
50. The method according to any one of Embodiments 1-45, comprising administering to the human patient 400 mg of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile twice a day.
51. The method according to any one of Embodiments 1-45 or 50, wherein the at least one compound is the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.
52. The method according to any one of Embodiments 1-45 or 50, wherein the at least one compound is the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.
53. The method according to any one of Embodiments 1-45 or 50, wherein the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.
54. The method according to any one of Embodiments 1-53, wherein the at least one compound is orally administered to the human patient.
55. The method according to any one of Embodiments 1-54, wherein the at least one compound is administered to the human patient in the form of at least one tablet.
56. The method according to any one of Embodiments 1-55, wherein the at least one compound is administered with water.
57. The method according to any one of Embodiments 1-56, wherein the at least one compound is administered with food.
58. The method according to any one of Embodiments 1-57, wherein the at least one compound is administered without food.
59. The method according to any one of Embodiments 1-58, wherein the human patient has had ITP for four or less years.

Claims or descriptions that include “or” or “and/or” between at least one members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all the group members are present in, employed in, or otherwise relevant to a given product or process.

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

The foregoing disclosure has been described in some detail by way of illustration and example, for purposes of clarity and understanding. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

What is claimed is:

1. A method for treating immune thrombocytopenia (ITP) in a human patient in need thereof who has not received prior treatment with a thrombopoietin receptor agonist (TPO-RA) or rituximab comprising administering to the human patient who has not received prior treatment with a TPO-RA or rituximab a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day for a treatment period, wherein the human patient in need thereof has at least one characteristic chosen from:
a. a platelet count of count <33,000/μL on two occasions no less than 7 days apart in the 15 days prior to beginning study treatment; and
b. a history of response (two or more platelet counts ≥50,000/μL with an increase of ≥20,000/μL) to at least one prior line of therapy.

2. A method for treating immune thrombocytopenia (ITP) in a human patient in need thereof who has not received prior treatment with a thrombopoietin receptor agonist (TPO-RA) comprising administering to the human patient who has not received prior treatment with a TPO-RA a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day for a treatment period, wherein the human patient in need thereof has at least one characteristic chosen from:
a. a platelet count of <33,000/μL on two occasions no less than 7 days apart in the 15 days prior to beginning study treatment; and
b. a history of response (two or more platelet counts ≥50,000/μL with an increase of ≥20,000/μL) to at least one prior line of therapy.

3. A method for treating immune thrombocytopenia (ITP) in a human patient in need thereof who has not received prior treatment with rituximab comprising administering to the human patient who has not received prior treatment with rituximab a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day for a treatment period, wherein the human patient in need thereof has at least one characteristic chosen from:
a. a platelet count of <33,000/μL on two occasions no less than 7 days apart in the 15 days prior to beginning study treatment; and
b. a history of response (two or more platelet counts ≥50,000/μL with an increase of ≥20,000/μL) to at least one prior line of therapy.

4. The method of claim 1 or 2, wherein the TPO-RAs are chosen from: recombinant thrombopoietin (rTPO), romiplostim, eltrombopag, and avatrombopag.

5. The method of any one of claims 1-2 or 4, wherein the patient has not received prior treatment with rituximab.

6. The method of any one of claims 1-5, wherein the at least one prior line of therapy, wherein at least one prior therapy is chosen from a splenectomy, intravenous immunoglobin (IVIG), corticosteroids, anti-D immunoglobulin therapy, and immunosuppressive drugs.

7. The method of any one of claims 1-6, wherein the patient achieves at least one platelet count of at least 20,000/μL above a baseline platelet count, wherein the baseline platelet count is the mean of two platelet counts taken on two occasions no less than 7 days apart in the 15 days prior to beginning treatment and a third count taken on the first day of the study.

8. The method of any one of claims 1-7, wherein the patient achieves at least two platelet counts of at least 20,000/μL above the baseline platelet count.

9. The method of claim 8, wherein the patient achieves at least two consecutive platelet counts of at least 20,000/μL above the baseline platelet count.

10. The method of any one of claims 8 or 9, wherein at least two consecutive platelet counts of at least 20,000/μL above the baseline platelet count are separated by at least five days, for example at least seven days.

11. The method of any one of claims 1-10, wherein the patient achieves a platelet count of at least 50,000/μL.

12. The method of any one of claims 1-11, wherein the patient achieves at least two platelet counts of at least 50,000/μL.

13. The method of claim 12, wherein the patient achieves at least two consecutive platelet counts of at least 50,000/μL.

14. The method of claims 12 or 13, wherein the at least two consecutive platelet counts of at least 50,000/μL are separated by at least five days, for example at least seven days.

15. The method of any one of claims 1-14, wherein the human patient achieves at least one platelet count of at least 50,000/μL within eight days of initiating treatment.

16. The method of any one of claims 11-15 wherein the human patient does not receive rescue medication during the four weeks prior to the most recent platelet count of at least 50,000/μL.

17. The method of any one of claims 1-16, wherein the human patient has had ITP for six or less years.

18. The method of any one of claims 1-17, wherein the human patient has a history of taking at least one prior ITP therapy prior to the start of the treatment period.

19. The method of any one of claims 1-18, wherein the human patient has a history of taking at least two prior ITP therapies prior to the start of the treatment period.

20. The method of any one of claims 1-19, wherein the human patient has a history of taking at least three prior ITP therapies prior to the start of the treatment period.

21. The method of any one of claims 1-20, wherein the human patient has a history of taking at least four prior ITP therapies prior to the start of the treatment period.

22. The method of any one of claims 1-21, wherein the human patient has a history of taking at least five prior ITP therapies prior to the start of the treatment period.

23. The method of any one of claims 1-22, wherein the human patient has platelet counts between 2,000/μL and 33,000/μL on two occasions no less than 7 days apart and within 15 days prior to the start of the treatment period.

24. The method of any one of claims 1-23, wherein the human patient had a splenectomy prior to the start of the treatment period.

25. The method of any one of claims 2 or 4-24, wherein the human patient has a history of taking rituximab prior to the start of the treatment period.

26. The method of any one of claims 3-25, wherein the human patient has a history of taking at least one TPO-RA prior to the start of the treatment period.

27. The method of claim 26, wherein the at least one TPO-RA is selected from rTPO, romiplostim, eltrombopag, and avatrombopag.

28. The method of any one of claims 1-27, wherein the patient has a history of response to at least one prior line of therapy, wherein at least one prior therapy is chosen from a splenectomy, intravenous immunoglobin (IVIG), corticosteroids, anti-D immunoglobulin therapy, and immunosuppressive drugs.

29. The method of any one of claims 1-28, wherein the human patient has a history of taking intravenous immunoglobin (IVIG) prior to the start of the treatment period.

30. The method of any one of claims 1-28, wherein the human patient has a history of taking corticosteroids prior to the start of the treatment period.

31. The method of any one of claims 1-28, wherein the human patient has a history of taking anti-D immunoglobulin therapy prior to the start of the treatment period.

32. The method of any one of claims 1-28, wherein the human patient has a history of taking at least one immunosuppressive drug prior to the start of the treatment period.

33. The method of claim 32, wherein the at least one immunosuppressive drug is selected from fostamatinib, mycophenolate mofetil (MMF), and cyclosporine.

34. The method of any one of claims 28-33, wherein the response to the prior ITP therapy comprised a platelet count of ≥50,000/μL.

35. The method of any one of claims 1-34, wherein the human patient has primary ITP.

36. The method of any one of claims 1-34, wherein the human patient has secondary ITP.

37. The method of any one of claims 1-34, wherein the human patient does not have chronic ITP.

38. The method of any one of claims 1-34, wherein the human patient has persistent ITP.

39. The method of any one of claims 1-34, wherein the human patient has chronic ITP.

40. The method of any one of claims 1-34, wherein the human patient has relapsing ITP.

41. The method of any one of claims 1-34, wherein the human patient has refractory ITP.

42. The method of any one of claims 1-41, wherein the treatment period is at least 8 days.

43. The method of any one of claims 1-42, wherein the treatment period is at least 28 days.

44. The method of any one of claims 1-43, wherein the treatment period is at least 84 days.

45. The method of any of claims 1-44, wherein the treatment period is at least 169 days.

46. The method of any one of claims 1-45, comprising administering to the human patient 400 mg of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof twice a day.

47. The method of any one of claims 1-46, wherein the at least one compound consists of at least one compound chosen from the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof.

48. The method of any one of claims 1-46, wherein the at least one compound consists of at least one compound chosen from the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile and pharmaceutically acceptable salts thereof.

49. The method of any one of claims 1-46, wherein the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or a pharmaceutically acceptable salt of the foregoing.

50. The method of any one of claims 1-45, comprising administering to the human patient 400 mg of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile twice a day.

51. The method of any one of claims 1-45 or 50, wherein the at least one compound is the (E) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.

52. The method of any one of claims 1-45 or 50, wherein the at least one compound is the (Z) isomer of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.

53. The method of any one of claims 1-45 or 50, wherein the at least one compound consists of a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile.

54. The method of any one of claims 1-53, wherein the at least one compound is orally administered to the human patient.

55. The method of any one of claims 1-54, wherein the at least one compound is administered to the human patient in the form of at least one tablet.

56. The method of any one of claims 1-55, wherein the at least one compound is administered with water.

57. The method of any one of claims 1-56, wherein the at least one compound is administered with food.

58. The method of any one of claims 1-57, wherein the at least one compound is administered without food.

59. The method of any one of claims 1-58, wherein the human patient has had ITP for four or less years.
